(19) United States
(12) Patent Application Publication
(10) Pub. No.: US 2016/0175164 A1
(43) Pub. Date: Jun. 23, 2016

(54) METHODS AND SYSTEMS FOR SCHEDULING PROCEDURES SUCH AS TOILETING

(71) Applicant: FRED BERGMAN HEALTHCARE PTY. LTD., New South Wales (AU)

(72) Inventors: Hadi Mashin-Chi, North Bondi (AU); Peter Curran, Oatley (AU); Peter Hubertus Aigner, Killara (AU)

(21) Appl. No.: 14/909,126

(22) PCT Filed: Jul. 30, 2014
(86) PCT No.: PCT/AU2014/000768

§ 371 Date: Jan. 31, 2016

(30) Foreign Application Priority Data
Jul. 31, 2013 (AU) 2013902853

(51) Int. Cl. A61F 13/42; G06Q 10/10; G06Q 50/22; G06F 19/00; G06N 3/12

(52) U.S. Cl. CPC A61F 13/42; G06F 19/3481; G06F 19/345; G06N 3/126; G06Q 50/22; G06Q 10/1097; G06F 19/3406; A61F 2013/425

(57) ABSTRACT

A method for determining one or more time indicators for scheduling one or more procedures includes receiving at a processing means one or more inputs comprising data relevant to the procedure, receiving at the processing means one or more objectives for optimising time indicators for performing instances of the procedure, and causing the processing means to receive as an input to an optimisation procedure at least a subset of the one or more received inputs and a representation of the one or more received objectives to produce one or more time indicators for scheduling the one or more procedures. The processing means generates outputs for representing one or more time indicators for scheduling the one or more procedures (such as toileting a patient) for which at least a subset of the received objectives are optimised.

### FIELD OF THE INVENTION

This invention relates to methods, systems and processes for determining points in time for taking a particular action. It relates particularly but not exclusively to methods and systems for determining a toileting schedule by identifying points in time when an individual should be toileted or, in the case of an incontinence event, undergo a wetness check or a pad change. It also relates to methods and systems for verifying a toileting schedule, and/or verifying one or more objective functions used to determine a toileting schedule, and to methods and systems for developing toileting awareness and toilet training.

### BACKGROUND OF THE INVENTION

Incontinence is a condition in which there is an uncontrolled release of discharges or evacuations from the bowel or bladder of an individual. Urinary incontinence refers to loss of bladder control resulting in involuntary or uncontrolled urination. Other forms of incontinence include faecal or bowel incontinence.

Because most sufferers of incontinence tend to be elderly or suffering from some form of disability a significant proportion of patients in care institutions such as hospitals, nursing homes, aged care facilities and geriatric institutions are sufferers of incontinence. Treatment options for incontinence can include behaviour management, medication and surgery. In circumstances where treatment is not available or unsuccessful the only option is to address the incontinence events themselves. Such methods include the subject wearing an incontinence aid such as an absorbent pad or diaper and/or making efforts to anticipate when a subject will experience an incontinence event, and take steps to assist with evacuation into a toilet.

Incontinence indicators and detection systems exist but they are, in most instances, rudimentary and merely alert a carer to the situation where an incontinence event has occurred and the subject requires a “pad change”. This can be undignified for the subject and to the extent that soiling may affect bed linen, clothing and the like, can be unhygienic, pose risks for skin integrity, and lead to significant demands on labour forces.

Additionally, without a reliable method for determining when a subject needs to evacuate into a toilet, attempts to toilet a subject may be unsupported due to rostering schedules that may be poorly matched to the subject's actual evacuation needs, giving rise to highly inefficient care. Furthermore, subjects who are generally immobile require the assistance of more than one carer to evacuate into a toilet. This imposes significant demands on labour which, if poorly matched to the subject's actual evacuation needs, may be doubly inefficient.

Somewhat related to the issue of incontinence is toilet training for small children. This phase in a child's life can be protracted and stressful for both child and parents or carers alike. There is often pressure and anxiety around toilet training and poorly coordinated actions by carers (such as parents, guardians, family members, child care workers and the like) can lead to frustration and upset. Critically, toilet training is most successful when the child has reached a level of developmental awareness. Efforts to toilet train before a child is “ready” can be wasted.

It would be desirable to provide a system capable of creating a care plan for subjects with incontinence that better meets their needs. It would also be desirable to provide a system capable of balancing the needs of the subjects with the people caring for them and other demands placed on those carers. It would also be desirable to provide a system that assists with toilet training, by determining when a child is developmentally ready to be trained, and to assist with toilet training.

### SUMMARY OF THE INVENTION

The present invention relates to systems and methods predominantly for use in care planning, particularly in relation to individuals suffering from incontinence, and for use in toilet training. Embodiments of the invention may be utilised as an adjunct to or in conjunction with existing or yet to be devised systems, methods and devices for monitoring incontinence, such as those disclosed in WO2007/128038 entitled “Moisture Monitoring System”, WO2011/054045 entitled “Improvements in Incontinence Monitoring and Assessment”, WO2011/156862 entitled “Apparatus and Method for Analysing Events from Sensor Data by Optimization” and WO 2013/003905 entitled “Improvements Relating to Event Detection Algorithms”, the entire contents of each of which are hereby incorporated herein by reference.

It is to be understood, however, that the principles of the invention are not limited to care planning and incontinence. Other areas of application include proactive monitoring and toilet training for infants and children, maintenance schedules for vehicles and machinery, aircraft landing schedules, scheduling for real-time taxi allocation to customers, livestock management, crop management and the like.

According to one embodiment, an aim of the invention is to provide information to carers including a set of time indicators that identify when an activity is likely (or unlikely) to occur. For example, the invention may provide a set of time indicators that identify when a subject is likely to experience a voiding event, in which there is partial or total evacuation of the bowel and/or bladder. Another aim may be to identify a set of time indicators that identify when an activity is unlikely, i.e. the subject is unlikely to experience a voiding event. That information can be used by a carer and more preferably by the automated system, to anticipate the event and, if appropriate, attempt to perform a “toileting procedure” with the subject over a toilet rather than have the evacuation occur into an incontinence aid, change the subject's incontinence aid or the like. An incontinence aid includes but is not limited to an item such as an absorbent pad, diaper, nappy, garment, dressing or the like used by individuals suffering from incontinence. Alternatively/additionally, the information may be used to identify periods of time where toileting procedures are not required. This in turn can be used to efficiently allocate carers and other resources, and activities such as outings or medical procedures that would otherwise be interrupted by a scheduled toileting procedure Embodiments of the invention may be used by the subject him/herself to achieve self-toileting, where the subject is mobile and alert but unable to anticipate, by themselves, when an evacuation (i.e. urinary and/or faecal voiding event) will occur.

Toilet Training is a skill that can only succeed once a subject is developmentally ready. Typically this requires the subject to be sufficiently responsive to cues and feedback from the environment, parents, carers and the like. In addition, the subject must be sufficiently self-aware that they can sense their own voiding events, urges and behaviour. Once they can sense these, they can begin to learn how to control them e.g. by “holding on” or going to the toilet, potty or the like.

Frustratingly, diaper technology has been so effective at drawing liquid away from the skin that it becomes more challenging for the subject to develop an awareness of voiding events that have occurred. Embodiments of the present invention help to resolve this issue by assisting in the development of the subject's awareness that a voiding event has occurred. Such assistance may be provided directly to the subject or via feedback first provided to the carer who then lets the subject know that there has been a voiding event.

The inventive methodology, as disclosed herein, helps to identify repetitive voiding profiles for individuals (observed in isolation or as part of a group of individuals e.g. in a care facility) typically by considering one or more n-hour periods of incontinence data collected for the individual/s. Incontinence data may be obtained manually, e.g. by a carer manually checking and changing incontinence aids, weighing the soiled aids and noting the time and relevant details (e.g. void type) of each event/check, as well as fluid and food intakes and other factors that may influence the incontinence behaviour of an individual. Alternatively/additionally, incontinence data may be obtained using sensors or other technology and may be supplemented (or supplied) by historical data for the subject including e.g. type of incontinence experienced, level of incontinence and the like. Ideally, each period of incontinence data utilised in the method corresponds to the same, or a similar, period of time in a n-hour block so that relevant events are monitored from which voiding patterns can be computed.

The inventive methodology is used to identify points or periods in time, that are temporally proximal to activities of interest, such as predicted voiding events, and which may be used for scheduling an appropriate procedure. In one embodiment, the inventive methodology is used to identify “toileting times” i.e. times for performing a toileting procedure. These toileting times may be used to alert a subject or carer so that the subject may be located on the toilet, commode or for voiding into another receptacle. In some embodiments, the invention may be used to identify a period of time where there will be no toileting procedures required, i.e. when the processing means determines that e.g. voiding events are unlikely to occur. For example for a particular subject one or more of the calculated time indicators designate no likely bladder or bowl evacuation and this is used to inform a carer that no toileting action will be required during those times. This has practical utility since toileting subjects into a toilet receptacle instead of an incontinence aid is more dignified for the subject and the carer, reduces the risk of skin integrity problems, and is more hygienic. Furthermore, the costs associated with incontinence aid usage and labour associated with pad changes is high, and may be reduced by use of the present invention. The costs associated with assisting a subject to and from the toilet may also be reduced by aligning the toileting activities with a likelihood of the subject being ready to evacuate their bladder and or bowel.

Thus, viewed from one aspect, the present invention provides a method for determining one or more time indicators for scheduling one or more procedures, the method including the steps of: receiving at a processing means one or more inputs comprising data relevant to the procedure; receiving at the processing means one or more objectives for optimising time indicators for performing instances of the procedure; and causing the processing means to receive as an input to an optimisation procedure at least a subset of the one or more received inputs and a representation of the one or more received objectives to produce one or more time indicators for scheduling the one or more procedures; wherein the processing means generates outputs for representing one or more time indicators for scheduling the one or more procedures for which at least a subset of the received objectives are optimised. The representation of the one or more time indicators may be used to cause display of the time indicators on a user interface, display device, print out or the like which may e.g. take the form of a toileting schedule. Alternatively/additionally, the output representation of the one or more time indicators may be used as inputs to other systems or modules and used to create e.g. staffing rosters, purchasing orders or the like.

Typically, the procedure is toileting a subject, and the inputs relate to the subject and his/her voiding behaviour including observation data relating to urinary and/or faecal voids, fluid and/or food intakes and other properties as discussed herein. Thus, the time indicators determined according to embodiments of the invention are used to designate a toileting schedule for the subject which enables efficient use of resources and time for attending to the subject's continence needs whilst also minimising unwarranted disruption, according to objectives that may be received as inputs to the system. In one embodiment the method includes causing the processing means to receive as an additional input an existing schedule of time indicators for performing one or more procedures, such as toileting a subject. The processing means is configured to perform an optimisation procedure to recalculate time indicators of the existing schedule to improve optimisation of at least a subset of the received objectives. This enables the “goodness” of the toileting schedule to be improved.

Preferably, the method includes causing the processing means to represent a received objective as an objective function. This may be done in any suitable manner such as by use of regression algorithms or the like. A time indicator may be a single point in time, a set of points in time, an interval specifying a time duration, a probability distribution in time or a function for calculating a time interval between time indicators.

In some embodiments, the one or more time indicators include activity time indicators which correspond to times when a voiding event is likely to be experienced by a subject. The activity time indicator may correspond with a voiding event type such as a faecal voiding event, a urinary voiding event or a combination of these, or the absence of a voiding event, indicating that voiding activity is unlikely for the associated time indicators. Alternatively/additionally, one or more time indicators may include toileting schedule time indicators used to define a toileting schedule for one or more subjects. The toileting schedule provides a plurality of time indicators for scheduling actions such as e.g. toileting a subject over a commode, bed pan, potty, receptacle or the like, refreshing or changing an incontinence aid worn by a subject, alerting the subject to self-toilet, or performing no specific toileting action (for periods during which no voiding activity is expected).

Typically, the inputs comprise data pertaining to one or more properties selected from a group including but not limited to event properties, intake properties, carer properties, subject properties, general properties, demographic properties and behavioural properties, to name a few. Objectives may be toileting objectives, a personal objective, a group objective, a financial objective, a workforce objective, a compliance objective, a carer objective, a productivity objective or any other objective many of which are described and/or exemplified described herein. Objectives may be specific to an individual subject or to a group or cohort of subjects. Alternatively/additionally objectives may be specific to a facility responsible for caring for one or more subjects and/or a carer.

In a preferred embodiment, the optimisation procedure includes causing the processing means to apply a multi-objective procedure in respect of a plurality of objective functions. The multi-objective procedure may include one or more of: combining n objective functions representing received objectives into m new objective functions where m≧n, reducing n objective functions representing received objectives into m new objective functions where m<n; and treating n objective functions representing received objectives separately and successively, depending on one or more of a rank order and a relative importance associated with each objective function. In some embodiments, a multi-objective procedure includes allocating a rank order to at least one of the objective functions to designate a rank order of importance of said at least one objective function relative to others of said objective functions. Alternatively/additionally, the multi-objective procedure includes the step of allocating a relative importance identifier to at least one of the objective functions representing an importance weighting or multiplier for said at least one objective function.

In some embodiments, the method includes causing the processing means to generate one or more objective functions suitable for a none-to-one carer to subject relationship (i.e. no carer), a one-to-one carer to subject relationship, a one-to-many carer to subject relationship, a many-to-one carer to subject relationship or many-to-many carer to subject relationship.

A one-to-one objective function may be derived by employing a multi-objective procedure in respect of a plurality of objective functions. These may include but are not limited to: a one-to-one distance function; a non-captured event objective function; and a non-preferred time objective function. A one-to one distance function represents an objective such as e.g. a risk of leakage objective, an unsuccessful toileting objective, a skin integrity objective, and an aid usage (consumption) objective to name a few.

A one-to-many objective function is derived by employing a multi-objective procedure in respect of a plurality of objective functions that may include one-to-one objective functions derived for each of the subjects in the one-to-many relationship, received objectives, if any, for the carer and one or more subjects in the one-to-many relationship, and/or a one-to-many collision avoidance objective function.

A many-to-one objective function may be derived by employing a multi-objective procedure in respect of a plurality of objective functions such as e.g. one-to-one objective functions derived for the subject in the many-to-one relationship, received objectives, if any, for one or more carers and subject in the many-to-one relationship, a carer-collision avoidance function and a carer-workload distribution function.

A many-to-many objective function may be derived by a employing multi-objective procedure on objective functions such as e.g. objective functions representing received objectives for individual ones of said carers and/or said subjects in said many-to-many relationship, and/or a one-to-many objective function or a many-to-one objective function as described above.

Typically, the multi-objective procedure includes an adjustment step causing the processing means to employ one or more functions or parameters selected from a group including but not limited to a distance function, a carer-collision avoidance function, a subject-collision avoidance function, a sparse time period rejection function, non-preferred times and a carer-workload function.

In an embodiment, the method further includes the step of causing the processing means to determine an optimum number of carers and/or subjects required to meet a satisfactory value of an objective function. This may involve causing the processing means to iteratively decrement automatically a total number of available carers and perform the multi-objective procedure until the satisfactory value is achieved for a many-to-one or many-to-many objective function; or to increment automatically the number of carers from one, and perform the multi-objective procedure until the satisfactory value is achieved for a many-to-one or many-to-many objective function. Alternatively, the processing means may decrement automatically a total number of subjects and perform the multi-objective procedure until the satisfactory value is achieved for a one-to-many or many-to-many objective function; or increment automatically the number of subjects from one, and perform the multi-objective procedure until the satisfactory value is achieved for a one-to-many or many-to-many objective function. In another embodiment of the inventive method, the number of subjects and/or carers can be optimised to change a subject-to-carer relationship type.

Ideally, the processing means is configurable to utilise one or more optimisation techniques such as parallel optimization, multi-level optimization or a hybrid of these. In parallel optimisation, a multi-objective procedure employs a plurality of objective functions, with or without rank order of importance and with or without relative importance weighting. In multi-level optimisation, there are multiple iterations of the optimisation procedure and each iteration corresponds to a level, the multi-level optimisation beginning with an initial iteration at a first level and ending after a final iteration at a final level, wherein the optimisation procedure optimises at each level a value of one or more objective functions applied at that level, while maintaining a value optimised from a previous level within an acceptable interval defined for said previous level.

In one embodiment, the method may include processing at least a subset of the received inputs to identify a relationship between a first input and a second input, and denoting the relationship between the first input and the second input as causal or non-causal. Thus, the optimisation procedure may further receive an input representing the causal or non-causal relationship between the inputs.

In a preferred embodiment, the processing means is configurable to generate outputs for displaying on a user interface a toileting schedule representing time indicators. The toileting schedule may include features such as (a) time indicators representing expected voiding event times (with or without certainty), (b) time indicators for performing a toileting procedure (with or without certainty or preference), (c) indicators representing expected voiding type and/or amount for an expected voiding event or toileting procedure (with or without certainty); (d) one or more carer identifiers (with or without preference) for performing a toileting procedure for a subject; (e) a subject identifier for performing a toileting procedure; (f) one or more expected voiding event types (with or without certainty); (g) one or more toileting procedure types (with or without preference); (h) one or more incontinence aid types and/or capacities (with or without user and/or carer preference); and (i) one or more locations for toileting (with or without user and/or carer preference). The incontinence aid type may or may not include liquid capacity and may or may not be associated with a preference for the subject or the carer. Similarly, the toileting location may or may not be associated with a preference belonging to either the subject or the carer. In any case, each of the features in a toileting schedule is determined by the processing means according to one or more objective functions.

Ideally, the processing means is configurable to outputs displayed on the user interface (e.g. in response to a filter selection input provided by a user), so that selected features of the toileting schedule may be hidden, visible, highlighted, emphasised or de-emphasised.

The invention also relates to verifying a set of time indicators, such as in a toileting schedule which has been created using the inventive techniques, or as may have been manually devised. The verification method may involve comparing at least a subset of time indicators in the toileting schedule with data representing a toileting schedule which is considered to be correct and verifying the time indicators as “correct”, where the difference is less than a threshold. Alternatively, the verification technique may involve calculating a value of a verification objective function applied to a set of time indicators in a toileting schedule, and verifying the time indicators as “correct” where the value of the objective function satisfies a threshold or range.

Another aspect of the invention relates to developing a subject's awareness of voiding events, as may be necessary for effective toilet training. The method may include detecting voiding events in a subject's diaper or incontinence aid e.g. using a sensor, and stimulating the subject to raise the subject's awareness of the voiding event having just occurred. Alternatively, the method may include determining one or more expected voiding event time indicators, ideally using the inventive method, and stimulating the subject, around the time that the voiding event is expected to occur. Stimulating the subject is intended to raise the subject's awareness, alerting them that a voiding event is imminent, has occurred or is occurring. The stimulation may be visible, audible or tactile, and may be delivered to the subject using a toy, light, speaker or body-worn device. Alternatively/additionally, stimulation may occur via a carer or parent who is alerted around the time of the expected voiding event, and attends to the subject by e.g. offering to take the subject to the toilet.

Viewed from another aspect, the present invention provides a method for determining a subject's readiness for toilet training, including determining one or more expected voiding event time indicators and automatically identifying a pattern in the subject's voiding behaviour, wherein a pattern in the subject's voiding behaviour indicates readiness for toilet training

In an embodiment, testing for a pattern in the subject's voiding behaviour includes determining if the value of a distance function (e.g. the sum of the distance between each time indicator and its nearest actual event, or an aggregate of the distances between each time indicator and its nearest actual event). Alternatively, a pattern may be identified by calculating time indicators for each of a series of time intervals in a period of observation, and wherein a pattern is confirmed if the difference between corresponding time indicators for each of said time intervals is less than a threshold. Alternatively, a pattern may be confirmed if the expected voiding event time indicators indicate a reduction in voiding frequency or expected voiding frequency, or when the frequency is less than a threshold for a given period of time.

In another embodiment, a pattern may be identified by first identifying a causal relationship between received input data representing observed voiding events (effect data) and one or more other received input data (cause data). A causal relationship may be identified by the processing means identifying a causality function representing a relationship between cause data and the effect data. A test value of the causality function is calculated using the expected effect data as inputs, and a real value of the causality function is calculated using observed effect data as inputs. The difference between the test value and the real value is calculated and a causal relationship is identified when the difference is less than a threshold.

Viewed from another aspect, the present invention provides a method for determining one or more time indicators for an expected event to occur, the method including the steps of: receiving at a processing means one or more inputs comprising data relevant to the expected event; receiving at the processing means one or more objectives for optimising the time indicators; and causing the processing means to receive as an input to an optimisation procedure at least a subset of the one or more received inputs and a representation of the one or more received objectives to produce one or more time indicators; wherein the processing means generates outputs for presenting on a user interface the one or more time indicators representing when an event is expected to occur and for which at least a subset of the received objectives are optimised.

Viewed from another aspect, the present invention provides a system for determining, automatically, one or more time indicators, the system comprising: (a) an input interface configured to receive: inputs comprising data relevant to calculation of the time indicators and one or more objectives for optimising said time indicators; (b) processing means configured to receive the inputs and the objectives and execute an optimisation procedure using at least a subset of the received inputs and a representation of one or more of the received objectives and to calculate values for representing one or more time indicators; wherein the optimisation procedure calculates the time indicators that optimise a value of one or more objective functions representing at least one received objective. Typically, the system includes an output interface communicatively coupled with the processing means and configured to receive a signal from the processing means for presenting on a display the time indicators calculated by the processing means.

Typically, the processing means is further configured to process a received objective and represent said received objective as an objective function. This may involve application by the processing means of a regression algorithm, or other techniques for devising the objective function. Alternatively, one or more objective functions themselves may be received as inputs.

In a preferred embodiment, the output interface is adapted to receive a filter selection, typically supplied by a user through a user interface, for filtering one or more features presented on the display. The filter selection is used to cause one more features of a display and particularly, of a toileting schedule presented on a display, to be hidden, made visible, highlighted, emphasised or de-emphasised.

A time indicator may be a single point in time, a set of points in time, an interval specifying a time duration between procedures, a function for calculating a time interval between procedures or a probability distribution in time. In some embodiments, one or more time indicators correspond to expected voiding events likely to be experienced by a subject. Time indicators representing expected voiding events may be used to define a toileting schedule for performing a toileting procedure for the subject. The processing means may further be configured to generate for one or more time indicators, a toileting procedure indicator for indicating the type of toileting procedure to be performed. Alternatively/additionally the processing means may be configured to calculate for one or more time indicators an expected voiding event-type indicator, indicating whether the voiding event is expected to be faecal or urinary or a combination of these.

Preferably, the processing means is configurable to perform a multi-objective procedure on a plurality of objective functions. The multi-objective procedure may involve one or more of (a) combining n objective functions representing received objectives into m new objective functions where m≧n; and (b) reducing n objective functions representing received objectives into m new objective functions where m<n; and (c) treating n objective functions representing received objectives separately and successively, depending on a hierarchical rank order of each objective function.

The processing means may be configured to perform optimisation procedures for a number of different carer-to-subject relationships, of the types described above. The processing means is configured to employ in the multi-objective procedure one or more functions or parameters such as a distance function; a carer-collision avoidance function; a subject-collision avoidance function; sparse time period rejection function; non-preferred times; a carer-workload function; and a non-captured event objective function. Further, the processing means may be configurable to employ one or more optimisation techniques such as parallel optimisation, multi-level optimisation and hybrid optimisation as described above.

Ideally, the input interface and output interface are communicatively coupled with one or more user-operable devices by a communication network including one or more wireless communication links.

It is to be understood that the inventive system may be adapted to perform steps corresponding to various ones or all of the methods described previously.

Viewed from another aspect, the present invention provides a non-transitory computer readable medium storing a computer program, the computer program causing a computer to execute a process for automatically determining one or more time indicators, the process including the steps of: (a) receiving at a processing means one or more inputs comprising data relevant to calculating the time indicators; (b) receiving at the processing means one or more objectives for optimising said time indicators; and (c) causing the processing means to execute an optimisation procedure on at least a subset of the one or more inputs and a representation of the one or more received objectives to produce one or more time indicators; and causing the processing means to calculate values for representing the one or more time indicators. Typically, the values are communicated to an output device. The process may also cause the processing means to calculate an objective function representing a received objective.

Typically, the one or more time indicators represent one or more expected voiding events for a subject. The process may further include causing the processing means to calculate a toileting schedule for performing a toileting procedure for a subject. A toileting procedure may involve e.g. toileting the subject over a commode or receptacle, refreshing or replacing an incontinence aid, or alerting the subject to self-toilet. Optionally, the toileting schedule may include one or more voiding-type indicators designating an expected voiding event as a type such as a faecal voiding event, a urinary voiding event; or a combination urinary/faecal voiding event.

The optimisation procedure executed by the processing means typically includes a multi-objective procedure for applying one or more functions or parameters derivable by the processing means, such as e.g. a distance function; a carer-collision avoidance function; a subject-collision avoidance function; sparse time period rejection function; non-preferred times; and a carer-workload function.

The non-transitory computer readable medium includes instructions causing the processing means to execute one or more optimisation techniques selected from the group including but not limited to: parallel optimisation, multi-level optimisation, and hybrid optimisation as described above. The non-transitory computer readable medium may further include instructions for generating a display signal for causing a display device to show a toileting schedule including assignment of a carer to a subject for each toileting procedure associated with a time indicator in the toileting schedule.

It is to be understood that the non-transitory computer readable medium may include instructions for performing steps according to the method described above, and the detailed description that follows.

### BRIEF DESCRIPTION OF DRAWINGS

FIG. 1 is a schematic illustration of a system according to an embodiment of the present invention.

FIG. 2 is a flowchart illustrating steps in a method for determining one or more points in time for performing an action, according to an embodiment of the invention.

FIG. 3 is a schematic illustration of schema for a toileting profile according to an embodiment of the invention.

FIG. 4 is an example of toileting schedule for a subject, derived according to an embodiment of the invention.

FIG. 5 illustrates three events sets obtained from a subject during an assessment period which may be used for determining a toileting schedule according to an embodiment of the invention.

FIG. 6 is a schematic illustration representing a “parallel” optimization procedure for use in deriving a toileting schedule according to an embodiment of the invention.

FIG. 7 is a schematic illustration representing a “multi-level” optimization procedure for use in deriving a toileting schedule according to an embodiment of the invention.

FIG. 8 is a schematic illustration representing a “hybrid” optimization procedure for use in deriving a toileting schedule according to an embodiment of the invention.

FIG. 9 represents a probability distribution of “time type” for a set of time indicators calculated according to an embodiment of the invention.

FIG. 10 shows curves representing visually, a probability of increase in risk of leakage according to a linear (L), S-shape (S) and square root (SR) type functions.

FIG. 11 is an example of a toileting profile showing how a non-preferred value may be considered in deriving a toileting profile.

FIG. 12 illustrates graphically, received input information used for determining a toileting schedule for a one-to-one carer-to-subject relationship according to an embodiment of the present invention.

FIG. 13 represents observation data for Subject 1 for use in an example of the invention.

FIG. 14 illustrates time indicators calculated for two aid changes PC-1 and PC-2 according to an example of the invention.

FIG. 15 illustrates optimal time indicators for three aid changes PC-1, PC-2 and PC-3 according to an example of the invention employing the same constraints as those applied in the example illustrated in FIG. 14.

FIGS. 16, 17 and 18 illustrate inputs received for each of Subjects 1, 2 and 3 respectively in a many-to-many carer to subject relationship.

FIGS. 19, 20 and 21 illustrate toileting schedules determined for each of Subjects 1, 2 and 3 respectively based on the inputs in FIGS. 16, 17 and 18, using a one-to-one procedure, according to an embodiment of the invention.

FIG. 22 illustrates a toileting schedule for Subjects 1, 2 and 3 optimised for two carers, according to an embodiment of the invention.

### DETAILED DESCRIPTION

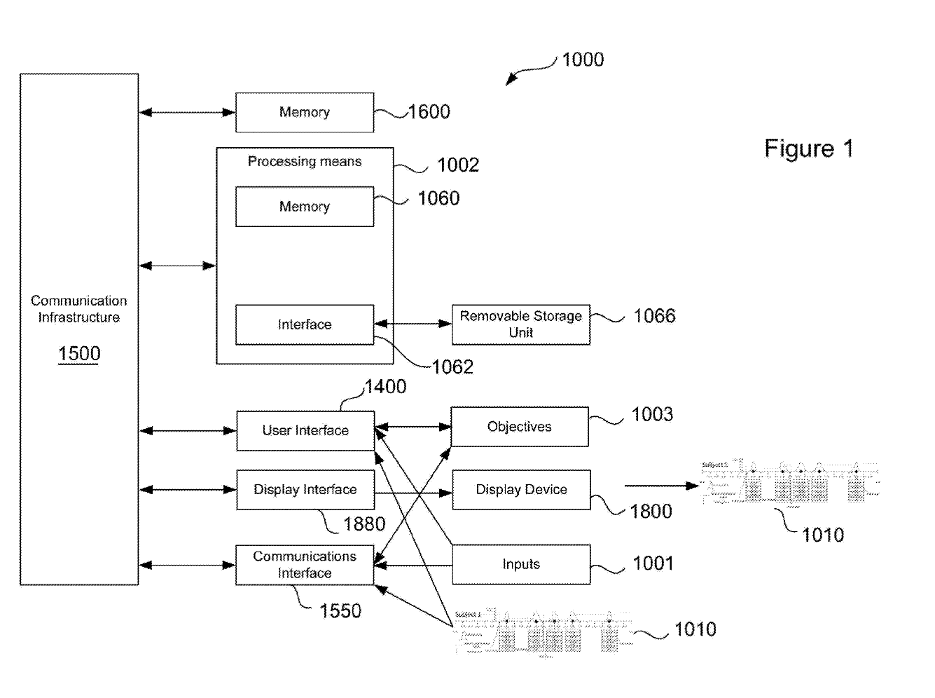

The invention will now be described by reference to the drawings and the several examples provided herein. FIG. 1 shows a system 1000 that may be utilised in performance of embodiments of the present invention. FIG. 1 shows a communications infrastructure 1500 connecting various elements of the inventive system which may be implemented over a communications network such as a LAN or a WAN. Inputs 1001 may be received through a communications interface 1550 as would be understood by one of skill in the art. Alternatively/additionally, inputs 1001 may be received by an input device such as a handheld or mobile computer operated by a user and coupled with the system via user interface 1400. Communications interface 1550 may also receive objectives 1003, and/or existing toileting schedules 1003, as may user interface 1400. Processing means 1002 has memory 1060 and may cooperate with a removable or remote storage unit 1066 via interface 1062. The system may have additional memory 1600 in the cloud or on another device directly or indirectly communicating with components of system 1000. Processing means 1000 is typically configured to receive one or more objectives 1003 and generate objective functions 1003a, although objective functions 1003a may be generated by other means and provided as inputs 1001. One or more display devices 1800 may couple with the system using wired or wireless means including communications interface 1550 and/or display interface 1880 to display time indicators and toileting schedules 1010 calculated according to embodiments of the invention. Toileting schedules 1010 may be received by other systems or methods or modules thereof for use in a range of different applications. Memory modules 1600, 1060 and 1066 may be used to store inputs 1001, objectives 1003, objective function 1003a, and toileting schedules 1010.

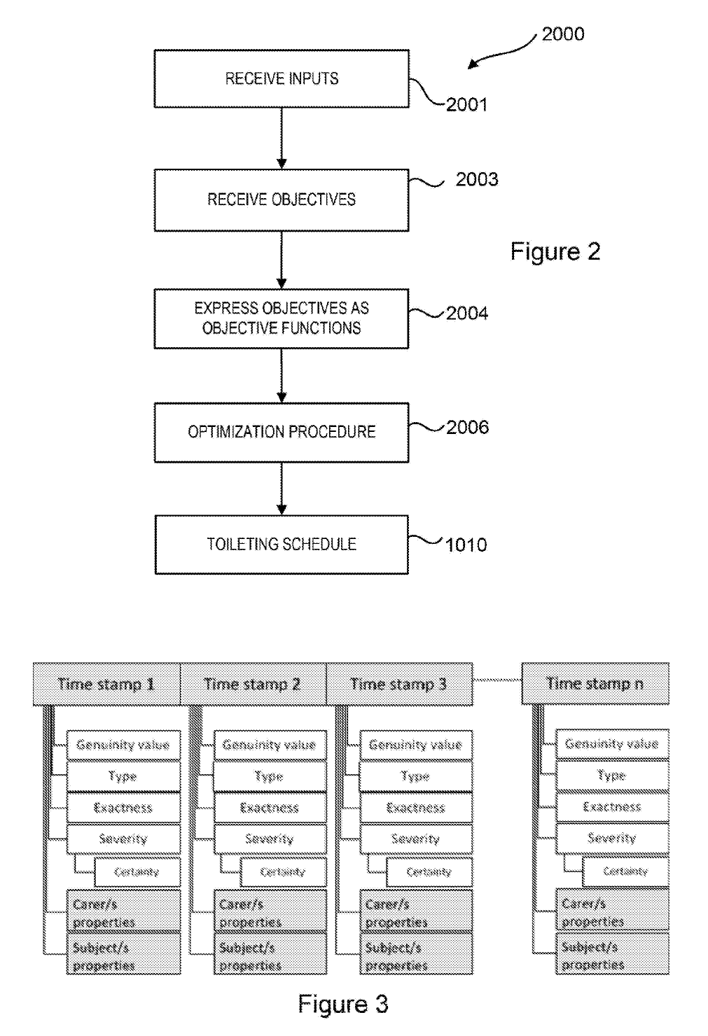

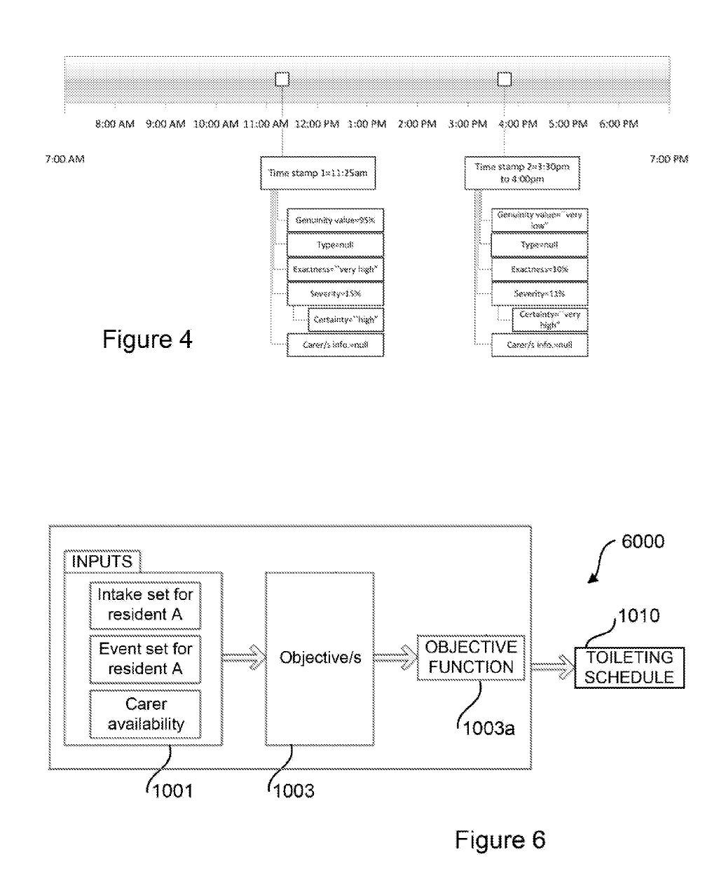

FIG. 2 is a flowchart illustrating steps in a method 2000 for determining one or more time indicators (points or periods in time) for scheduling a procedure. The procedure may involve taking an action such as toileting a subject, performing a continence aid check or change, or e.g. reminding the subject to go to the toilet. In an embodiment, the one or more time indicators indicate when an activity such as a voiding event is expected to occur. Time indicators may have attributes, such as but not limited to: type of event, estimated size or volume of the event, type of action to be taken, and identity of a carer to take the action. An example is given in FIG. 3. A toileting schedule created according to embodiments of the invention may comprise time indicators representing expected voiding events/activities, and/or it may comprise different time indicators which precede or follow time indicators for expected events, depending on the procedure to be performed. For example, a toileting procedure that involves taking a subject to the toilet is scheduled at time indicators that precede the time indicators for the expected events, whereas a toileting procedure that involves changing an incontinence aid is scheduled after a time indicator for the expected event. In a preferred embodiment, a user may select a display filter that enables presentation of selected types of time indicators, and hides, highlights, emphasises or de-emphasises others. For example, filter may display time indicators for performing toileting procedures, but hide the time indicators representing when a voiding event is expected to occur, or vice versa.

FIG. 2 shows steps in a method for determining a toileting schedule for a subject in a one-to-one carer-to-subject scenario. One-to-many, many-to-one, and many-to-many carer-to-subject scenarios may be treated similarly, where the term “many” is to be taken as meaning two or more.

In a step 2001, one or more inputs 1001 are received, ideally via a communications interface, at processing means 1002. The inputs 1001 consist of input information selected from a range of information types that are useful for defining a toileting schedule. The input information may include but are not limited to: event properties (e.g. for events in event sets), intake properties (e.g. for intakes in intake sets), carer properties, subject properties and properties relating to general information such as a facility where a subject is located and the location of toilets within that facility. Table 3 is an example of how the input information may be represented. Further details regarding information received by processing means 1002 as inputs 1001 are provided below.

In a step 2003, one or more objectives 1003 are received at processing means 1002. The objectives are typically determined according to one or more goals of the toileting schedule and may include one or more of the objectives identified in Table 1. These objectives may also be used to verify the effectiveness of a toileting schedule. A toileting schedule may be derived according to embodiments of the invention. The objectives may also be used to measure the correctness of, and/or improve an objective function used to determine a toileting schedule according to embodiments of the present invention. In an embodiment, a user may select or supply objectives for deriving or verifying the toileting schedule. Selection may be from a list presented on a display or other means. Selectable objectives may include but are not limited to objectives identified in Table 1. A user may also select features for calculation such as a toileting schedule, a schedule of expected voiding event times, a schedule of times during which voiding is unlikely, and various other features as discussed herein, or a combination of these. One or more features may be highlighted, emphasised, de-emphasised, hidden or moved on a display, under control of an authorised user (or any user) using a device such as a keyboard, mouse, touchscreen, stylus or the like.

TABLE 1

| | | Value of Objective | Value of Objective |
|---|---|---|---|
| Ob- | | Function for frequent | Function for |
| jective | Description | toileting or pad | infrequent toileting |
| No. | of Objective | changing | or pad changing |
| 1 | Risk of | Minimized | Maximized |
| | leakage | | |
| 2 | Unsuccessful | Minimized or maximized | Minimized or |
| | toileting | (depending on definition | maximized |
| | | of unsuccessful toileting) | (depending on the |
| | | Amount of urine voided | definition of |
| | | into toilet vs percentage | unsuccessful toileting) |
| | | of a toileting event being | Amount of urine |
| | | successful | voided into toilet |
| | | | (e.g. in percentage |
| | | | of a toileting event |
| | | | being successful |
| 3 | Skin | Minimized | Maximized |
| | problems | | |
| 4 | Carer | Maximized | Minimized |
| | workload | | |
| 5 | Pad usage | Maximized | Minimized |
| 6 | Subject | Maximized or minimized | Maximized or |
| | comfort | (depending on the | minimized (depending |
| | | definition of comfort and | on the definition of |
| | | dignity) | comfort and dignity) |
| 7 | Compliance | Maximized or minimized | Maximized or |
| | with | (depending on | minimized (depending |
| | regulations | regulations) | on regulations) |
| 8 | Risk of fall | Minimized | Maximized |
| 9 | Resource | Maximized or minimized | Maximized or |
| | consumption | (depending on the | minimized (depending |
| | | practice of a facility) | on the practice of a |
| | | | facility) |

Objectives 1003 received by processing means 1002 may be determined in any suitable manner. The objectives and their importance may be determined based on facility related requirements and/or interests. In an embodiment the facility related requirements/interests may be captured by feedback collected from e.g. carers, facility management and subjects themselves and supplied to a system via Interface 1550 which communicates with processing means 1002 via system communications infrastructure 1500. The collected feedback typically contains information on which objectives need to be considered for determining a toileting schedule and how important these objectives are. These feedback results may take any suitable form such as e.g. “ranked form” or “relative importance form” or a hybrid of these.

For example, feedback results in “relative importance form” may be used to ascertain the relative importance of one or more objectives, relative to other objectives. For example, the objective “reducing the risk of leakage” may be twice as importance as the objective “minimizing risk of leakage”. Further, the objective “minimizing risk of leakage” may be identified according to the feedback results as being four times as important as the objective “maximizing compliance with care guidelines”. A numeric or linguistic identifier may be assigned to each objective to indicate its relative or ranked importance. See for example Table 2. This “relative importance” feedback results form may be used in “parallel-level” or “hybrid” optimization procedures discussed below.

TABLE 2

| Objective | Relative Importance |
|---|---|
| Minimizing the risk of leakage | 8 |
| Minimizing the aid usage | 4 |
| Maximizing compliance with regulations | 1 |

Alternatively, feedback results may be in “ranked form” and used to ascertain a rank order of objectives. Thus, in “ranked form”, objectives are related in an order of importance, e.g. first (most important): “minimizing risk of leakage”; second: “minimizing pad usage”; third: “maximizing compliance with care guidelines”; and so on. In a variation of ranked form, one or more objectives may have equal importance, e.g. equal first: “minimizing the risk of leakage” and “minimizing aid usage”; and second: “maximizing compliance with care guidelines”. In “ranked form” the relative importance may not be available or may be inaccurate. This feedback results form may be used in “multi-level” or “hybrid” optimization procedures discussed below.

Alternatively/additionally, the feedback results may take a hybrid form, combining both “ranked” form and “relative importance” form. For example, the objective “minimizing risk of leakage” may be twice as important as the objective “minimizing pad usage”, and “maximizing compliance with care guidelines” has lower rank than “minimizing pad usage”. This hybrid form of feedback result may be used in “multi-level”, “parallel-level” or “hybrid” optimization procedures discussed below.

Typically, received objectives are expressed mathematically, in the form of objective functions. This is achieved in a step 2004. Additionally/Alternatively, processing means 1002 may improve an existing objective function 1003a by receiving an existing toileting schedule, determining the “goodness” of its performance in meeting the objectives (and objective functions) used to create it, and modifying parameters of the objective function to improve performance. Throughout this specification, the terms “objective” and “objective functions” may be used interchangeably.

In a step 2004, where more than one objective is received at processing means 1002, the objectives 1003 are treated by a “multi-objective procedure”. A multi-objective procedure may involve e.g. a “combination procedure”, a “reduction procedure”, “hierarchical ranking procedure”, a combination of these or other suitable procedures aimed at dealing with optimising time indicators for more than one objective. In an embodiment, the hierarchical ranking procedure treats one or more objectives separately and successively depending on a hierarchical rank order of each objective (as may be obtained by “ranked form” feedback as discussed above). In an embodiment, the reduction procedure combines objectives such that there are fewer objective functions 1003a than the number of received objectives 1003. In a combination procedure, there may be more or the same number of objective functions 1003a as the number of received objectives 1003. A reduction or combination procedure may utilise the relative importance (e.g. weighting multiplier) of one or more objectives 1003 when generating objective functions 1003a. In an embodiment, two or more objectives 1003 with the same rank or the same relative importance may be blended in any of a combination, reduction or hierarchical procedure to generate objective functions 1003a.

In a step 2006, a toileting schedule is determined by processing means 1002 using an optimisation procedure which optimises the value of the objective functions 1003a. This may be achieved by determining e.g. points in time when value of the objective function 1003a is optimised. Alternatively, the optimisation procedure at 2006 may determine a function Rf( ) (such as a relax-period-of-time-function) for calculating a point in time pᵢ where i is the point in time index. Rf( ) receives one or more inputs to calculate initial time indicator p₁ representing when a voiding event is likely and which is then used to determine when a procedure is to be performed. The procedure may be toileting a subject or alerting the subject to self-toilet. These procedures must be done prior to the point in time pᵢ. Alternatively, the procedure may be changing an incontinence pad which may be scheduled to occur around the indicator pᵢ. The calculated point in time, p₁, is then used, together with one or more further inputs, for calculating the next point in time p₂ for determining when the next procedure should be scheduled. The process continues, utilising the function Rf( ) for calculating subsequent time indicators for determining when an activity (e.g. voiding) is likely to occur from which time indicators for scheduling toileting procedures may be determined.

In an alternative embodiment, optimisation procedure 2006 may involve receiving at processor 1002 the inputs 1001 and objectives 1003 as well as an existing toileting schedule 1010, which has been previously determined. The existing toileting schedule 1010 may have been derived using the inventive method 2000, or using some other method, or derived manually by observing the behaviour of the subject (or of more than one subject).

If the conditions used to determine a toileting schedule change, for example if information corresponding to the inputs is removed, added to or updated, the toileting schedule 1010 may be revised by re-executing the method 2000 using the changed inputs 1001 and objectives 1003, or by re-executing the method 2000 using the changed inputs 1001, objectives 1003 and the toileting schedule 1010 from a previous iteration of the method. Manual adjustment i.e. by carers modifying the current toileting schedule may also be permitted in some circumstances using user interface 1400.

In another embodiment, if one or more inputs 1001 change, the impact of the changes can be calculated by processing means 1000 according to how recently the change occurred. For example, say a toileting schedule 1010 was determined for a subject in January 2011 based on inputs 1001 including incontinence data in the form of an events set captured in the same month. In February 2011, a second events set is obtained from the subject. Both events sets may be utilised as inputs 1001 in the method 2000 (which may, in an embodiment, utilise a toileting schedule 1010 previously calculated according to method 2000) for calculating an updated toileting schedule 1010.

However, it may be desirable for the more recent February events set to have greater impact on the schedule than the January events set. This may be achieved by increasing the value of the objective functions 1003a according to the recency of the input, i.e. increasing the value of the objective functions obtained for the more recent February data. The optimization procedure 2006 is then applied on the modified values obtained using the objective functions which. The optimization procedure, typically through many iterations, identifies time indicators that optimise (maximise or minimise) the modified values of the objective functions 1003a as desired to meet the various objectives.

Alternatively, the influence of inputs comprising previously collected incontinence data may be the same regardless of how recent the data. Other factors such as data quality or certainty may be used to differentiate data sets relied upon in deriving a toileting schedule, where the impact of high quality data and data having better certainty is enhanced by increasing the value of the objective functions obtained for the higher quality and better certainty data.

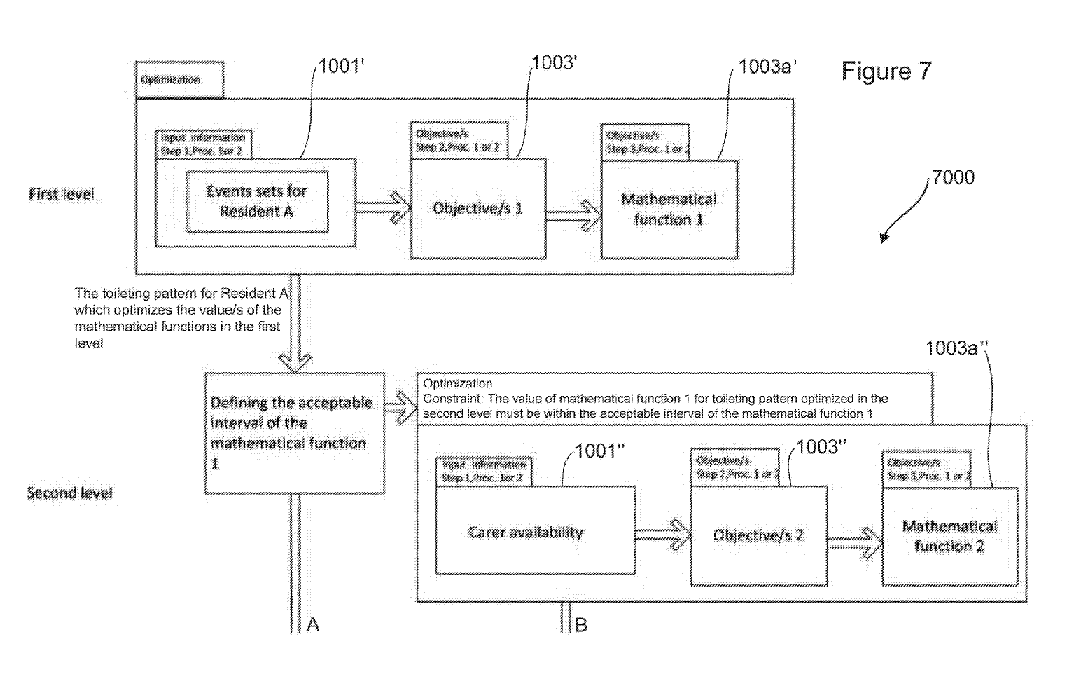

An example showing application of the method 2000 for determining a toileting schedule 1010 will be described by reference to FIG. 5. Meanwhile, further detail pertaining to the properties of inputs 1001, objectives 1003 and toileting schedules 1010 are provided below.

### Inputs 1001

Input information supplied as inputs 1001 received by processing means 1002 may include properties of an observed event (e.g. an incontinence event observed manually or using sensors or the like), or properties of a plurality or set of observed events (events sets). Alternatively/additionally, inputs 1001 may comprise properties representing time intervals during which incontinence events have not occurred; properties of an intake such as fluid, food or medication consumed by the subject by mouth or otherwise; and/or properties of a plurality or set of intakes (intakes sets). If one or more event properties (such as occurrence, time, type, size etc.) correlate with e.g. an intake event then a causal relationship may be drawn. A causal relationship may be defined mathematically by the system and utilised by the processing means 1000 in a Causality Test employed in e.g. toilet training systems or to diagnose the type of incontinence experienced by the subject. This may in turn be used to suggest a treatment or management regime e.g. by interrogating a lookup table of related incontinence types and treatment/management regimes stored in memory 1060 associated with or accessible by processing means 1000.

For example, if the input data represents a sneeze or cough at a time indicator which is the same as or close to a urinary voiding event (and ideally a pattern of this behaviour), the system may be adapted to determine the existence of a causal relationship between the two, and identify the incontinence type for that subject as Stress or Transient incontinence. The system may further be adapted to provide, automatically, a recommendation that the subject perform pelvic floor muscle exercises to improve their condition.

In another example, if the input data represents fluid intake at a time indicator which is the same as or closely followed by a urinary voiding event (and ideally a pattern of this behaviour), the system may be adapted to determine the existence of a causal relationship between the two, and identify the incontinence type for that subject as Reflex incontinence.

In cases where the goal is to determine time indicators that represent likely voiding event times arising from usual toileting behaviour, it may be desirable to pre-process or filter the input data to exclude events that have been identified as e.g. Stress, Transient or Reflex incontinence events since those events are triggered by an action such as coughing or sneezing which may occur at random times or time intervals. Where a causal relationship is identified to exist between inputs, it may be desirable that inputs 1001 captured by processing means 1002 include properties of the event or events set as well as properties of the intake or intakes set causing the event or events set. Table 3 sets out non-limiting examples of input types and how they may be represented, according to embodiments of the present invention.

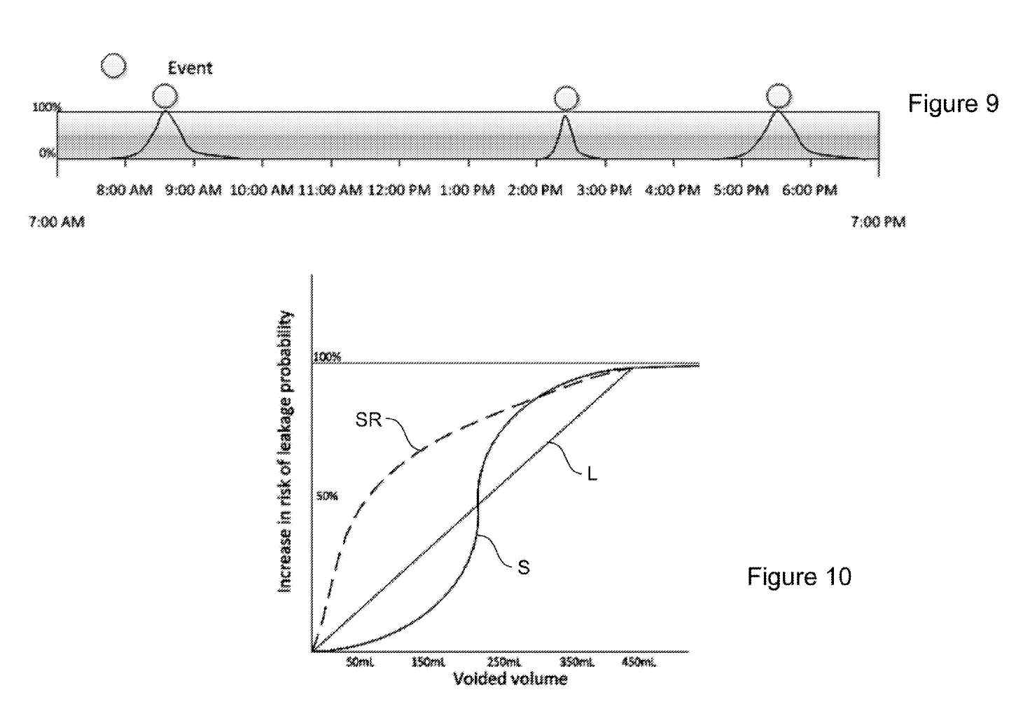

TABLE 3

| Input type | Type | Example(s) |
|---|---|---|
| Time | Exact | 10:22 am |
| | Interval | 10:18 am to 10:28am |
| | Category | Early morning; evening |
| | Probability | See FIG. 9 |
| | distribution | |
| Location | Numeric | Global Location Number (GLN)/coordinate |
| | Interval | Approximation to a GLN/GPS |
| | Linguistic | Room 3; wing 4; level 4; lounge |
| Ordinary | Numeric | 4; 5; 12; 3600 |
| | Interval | a to b; 20% to 30% |
| | Probability | 0%, 50%; 100%, |
| | Linguistic variable | low; medium; high |

FIG. 9 represents a probability distribution of a “time type” input for estimating the time of an event activity which may be used to determine time indicator scheduling a procedure such as toileting a subject, calculated according to an embodiment of the invention. Where the value of the probability curve approaches 100%, the likelihood of an event occurring at that time is greater. An input of any type may also have a “certainty” value to indicate how confident the system is in the correctness of the input.

“Ordinary” type inputs may be used for representing non-time and non-location type input information such as expected event size, or probability of correctness. Ordinary type data may be represented using e.g. exact numbers, numeric intervals, probability values, probability distributions, linguistic variables (e.g. highly likely, not likely) and the like, or a combination of these. By way of example, received input data may be used to estimate that an incontinence event expected to occur at a particular time has a 20% likelihood of being faecal matter and “very unlikely” to contain urine; or is expected to be 20% faecal matter and 80% urine.

If inputs comprising one or more observed event properties (such as event occurrence, time, type, size etc.) correlate with e.g. an observed intake then a causal relationship may be drawn. In this situation, it may be desirable that inputs 1001 captured by processing means 1002 include properties of the event or events set as well as properties of the intake or intakes set causing the event or events set. In a special case that may be particularly useful for deriving a faecal event toileting schedule, if one or more of the occurrence, time, type and size of observed faecal events occur with what may be regarded as a causal relationship with one or more intake (e.g. the intakes and observed faecal events occur close together in time) then it is desirable that inputs 1001 received by processing means 1002 include properties of the faecal event/events set and/or properties of the related intake or intakes set. Similarly, for deriving a urinary event toileting schedule, if one or more of the occurrence, time, type, size of observed urinary events occur with what may be regarded as a causal relationship with one or more intakes (e.g. the intake events and observed urinary events occur close together in time) then it is desirable that inputs 1001 captured by processing means 1002 include properties of the urinary event/events set and/or properties of the intake or intakes set.

In some instances, input information may be inaccurate, e.g. where one or more properties of one or more events, intakes, carers, subjects or general information is missing, insufficient or inconsistent. Time periods corresponding to inaccurate input information may be identified as such e.g. by labelling the inputs as a “sparse period of time”. A sparse period of time input may be grouped as e.g.:
Type 1: where there is insufficient data provided with inputs comprising one or more intakes, events, carers, subjects or general information to derive an acceptable toileting schedule;
Type 2: where data for one or more intakes, events, carers, subjects or general information which is necessary or beneficial for deriving a toileting schedule is missing from the received input; and
Type 3: where the input comprises data for one or more intakes, events, carers, subjects or general information is inaccurate, uncertain or inconsistent, and an acceptable toileting schedule cannot be derived.

Data received as an input may have different properties. Table 4 sets outs non-limiting examples of input properties for different categories of inputs and how they may be represented using input types, according to embodiments of the present invention.

TABLE 4

| Input | | |
|---|---|---|
| category | Property | Input type |
| Event | Faecal; urinary | Ordinary |
| | Size of event | Ordinary |
| | Time of event occurrence | Time |
| | Date of event occurrence | Time |
| Intake | Meal | Ordinary |
| | Fluid | Ordinary |
| | Medication | Ordinary |
| | Time/date of intake | Time |
| | Intake size | Ordinary |
| | Workload | Ordinary |
| Carer | Location e.g. during particular times/shifts | Location |
| | Performance (accuracy, speed) | Ordinary |
| | Non-preferred toileting time (e.g. break, sick leave) | Time |
| | Degree of non-preference | Ordinary |
| Subject | Physical: mobility, medication, diet, skin, weight, | Ordinary |
| | morbidity | |
| | Incontinence type (urge, stress, mixed, overflow, | Ordinary |
| | functional, reflex and transient), severity | Ordinary |
| | Mental condition | Time |
| | Duration of assisted toileting | Time |
| | Non-preferred Toileting time (e.g. meals, sleeping) | Ordinary |
| | Degree of non-preference | Location |
| | Location e.g. at particular time of day | Ordinary |
| | Continence holding ability | Ordinary |
| General | Toileting frequency: number permitted per day per | Ordinary |
| | subject; may depend on time and/or other properties | |
| | Time period allowable between toileting events | Time |
| | Non-preferred toileting time (e.g. medication time) | Time |
| | Toilet locations | Location |
| Sparse | Start and end time | Time |
| period of | Duration | Time |
| time | Severity of sparse period | Ordinary |

The “severity” of a sparse period of time may be determined according to e.g. the number of events and/or intakes in that period of time and optionally their certainty; or according to one or more properties of one or more events/intakes in the events sets/intakes sets. Input properties may also have a “certainty” which is of type of “ordinary” and is used to indicate how confident the system is in the correctness or of the data. Certainty may be designated by a value (e.g. 1 represents 100% certainty; 4 represents 10% certainty) or by a linguistic identifier, rank order, function or the like.

A pre-determined toileting schedule may also be received as an input. Properties of a toileting schedule 1010 may be specified in terms of e.g. properties of expected events, intakes, carers, subjects, general information, sparse periods, toileting activities related to expected events and the like. The properties and time indicators for events and/or toileting activities in the schedule may be influenced by an individual interacting with the system and/or by information drawn from the literature, research or other systems and automatically influencing the schedule. The properties of a toileting schedule may be defined in any data structure suitable for storing, retrieving, representing and processing using the inventive system. Values of any input properties not provided may be stored as a NULL value, may be deleted, or treated by a combination of these depending on how the system is designed.

In another embodiment, inputs may be used in a method for developing a subject's awareness of his or own voiding events as may be useful e.g. in preparation for toilet training. This may first involve ascertaining a subject's awareness of his or her own voiding activity. This may involve e.g. monitoring a subject's facial expressions (e.g. crying, closing eyes, and so on) or body movements (squirming, shuddering, holding genital area, and so on) during a voiding event. Detection of facial expressions and/or body movements may be automatic, e.g. using cameras capturing images and using facial expression analysis algorithms and the like to associate captured images of particular expressions with a voiding event that has occurred. Similarly, body movements may be detected using cameras and/or pressure sensors, accelerometers, gyroscopes, electromyogram sensing or the like. Alternatively/additionally, a carer may observe the subject and their behaviour in response to voiding. Voiding may be known to have occurred by use of e.g. wetness signal data derived from a sensor in the subject's diaper, a colour changing diaper or the like.

If the subject does not have an established awareness of their own voiding behaviour, it is unlikely the subject is ready for toilet training. However, using inventive techniques, readiness for toilet training can be developed using embodiments of the present invention, particularly those that provide methods for determining time indicators for expected voiding events. Toilet training readiness may be developed by providing the subject with a form of stimulation just prior to and/or during and/or just after a voiding event is expected to occur. The stimulation may be in the form of a human voice e.g. a recording or computer generated voice asking if the subject needs to go to the toilet, or uttering the name of the type of voiding event expected. This not only helps with developing awareness of voiding, but may also develop the subject's language skills and hence ability to express toilet training related concepts. In other arrangements, the stimulation may be relatively simple and involve actuation of e.g. a light, sound and/or vibration or the like.

In another embodiment, the subject is provided an incentive if s/he performs a movement or activity, or asks for assistance to be toileted before voiding occurs. Such incentive may be of any suitable type, such as e.g. a fun sound, a toy to play with, music, a story, food, or other offerings valued by the subject.

A subject may be identified as ready for toileting when their awareness of their own voiding behaviour is adequately developed. This can in many instances be ascertained by the presence of a pattern in the subject's voiding behaviour and may be tested using inventive techniques aimed at identifying the presence of a pattern in the subject's voiding behaviour. A number of pattern tests have been conceived, as discussed below. A one-to-one distance function as employed in Pattern Test A measures the sum of the distances of each time indicator to its nearest actual event. Alternatively, the distances of each time indicator to its nearest actual event may be aggregated with other input information by a suitable aggregate function.

Pattern Test A:
Determine expected event time indictors for a subject for a period of time, e.g. 3 days;
   IF the expected event time indictors have a one-to-one distance function value less than an acceptable threshold
   THEN a pattern is identified.

Pattern Test B:
Determine expected event time indictors for a subject for a period of time, e.g. 3 days;
Split the period of time into intervals, e.g. three 1-day intervals;
Determine expected event time indictors for the subject for each of the intervals;
   IF the difference between expected event time indictors for each of the intervals is less than an acceptable threshold;
   THEN a pattern is identified.

Pattern Test C:
IF the frequency of voiding has decreased or is less than an acceptable threshold;
THEN a pattern is identified.

Pattern Test D:
IF a Causality Test determines there is a causal link between one or more input data and the observed voiding event input data;
THEN a pattern is identified.

Causality Test:
Receive one or more sets of input data, referred to as Cause data;
Receive one or more sets of input data, referred to as Effect data;
Determine if there exists a function e.g. C( ) that can map one or more properties of the Effect data from one or more properties of the Cause data.
   IF the difference between the value of C( ) for properties of the estimated Effect data and the value for C( ) for actual properties of the Effect data is less than a threshold
THEN there exists a causal relationship between Cause data and Effect Data

For example, the Causality Test may be used to determine a causal relationship between fluid and food intake “Cause data” received as inputs and observed voiding event “Effect data” received as inputs, each of which include time stamps. Alternatively a causal relationship may be identified between “Cause data” representing a body activity (such as squirming, squatting or holding the genital area) or facial expressions, crying etc. and “Effect data” representing actual voiding events.

The results from one or more of the Pattern Tests and the Causality Test may be used for awareness development, voiding awareness or readiness testing, in toilet training, identifying incontinence type (urge, stress, mixed, overflow, functional, reflex and transient) and/or in incontinence management. Further, a causal relationship, which may be determined using e.g. a Causality Test, between subject movement “Cause data” and voiding event “Effect data” and associated time stamps may be utilised in methods and systems for awareness development according to embodiments of the invention. Awareness development may further involve conditioning and behavioural modification through e.g. positive reinforcement or the like which may be delivered by or with the aid of features of the inventive system.

If the outcome of one or more Pattern tests and/or a Causality Test indicates the existence of a pattern, then in an embodiment, the subject has completed the awareness development phase in preparation for toilet training, and is ready to be trained. In another embodiment, if a Causality Test finds a causal relationship between subject movement and voiding activity data, the awareness development phase is completed. Any combination of Pattern Tests and/or Causality Tests may be used to determine a subject's preparedness to commence toilet training.

A Toilet Training system according to some embodiments of the present invention may comprise a feedback device to provide feedback to the subject before or during the occurrence of a void. This may offer the subject with any feedback suitable for raising their awareness of the occurrence or imminence of a voiding event. In some embodiments, the feedback provided or controlled by the feedback device comprises a human voice (which may for example be the carer's recorded voice) uttering the name of the type of void occurring. This may assist the subject not only to develop an awareness of the voiding behaviour but also to develop an understanding of the concept of voiding and the language associated with it. Other forms of feedback may be provided using the feedback device, for example a light, sound or vibration, etc. In some preferred embodiments, feedback is provided by the carer in response to toilet training information communicated to the carer from the processing means that calculates time indicators representing expected voiding times.

In various embodiments, a subject may be given an incentives during toilet training, e.g. to control the urge to void by “holding on”. “Holding on” success may be established if the subject has controlled the urge to void by delaying a voiding activity expected to occur (according to a time indicator calculated by the processing means). Incentives may include e.g. a food reward, a favourite toy to play with, music, a sound, story, or any other suitable reward for successes in toilet training.

In some embodiments, the inventive toilet training system monitors the period during which there has been no voiding activity and creates an alert for the carer when the period is sufficiently long so as to create a high probability that a void is imminent. At such time the carer can then take the subject to the toilet so that they can void in the appropriate place (e.g. toilet or potty) and receive positive feedback for this. The alert may be received by the carer e.g. by a hand held computer, smart phone, tablet device or the like as are known in the art, and which, in order to interoperate with components of the inventive systems disclosed herein, may be installed with an application or similar software that renders the device suitable for receiving signals that alert the carer in the various manners disclosed herein. Alternatively, the system may send a similar alert to the subject e.g. using computer, a hand held tablet or gaming device, electronic toy or the like.

In some embodiments the system provides incentives for the subject to control voiding by “holding on” for successively increasing periods of time so as to train the subject to gain greater control over their bladder and/or bowel and voiding urges. Incentives may be provided in any suitable way and may involve feedback/alert systems of the kind described above. In some embodiments colourful charting on a computer or tablet device may provide a “happy face”, star, zoo animal or other positive icon to reward the subject for holding on or other successful toileting behaviour. Conversely, a “sad face” or other less positive symbols or icons may be employed when the subject has not met the ideal holding on time or other goal during toilet training. Ideally, the subject may be praised and/or rewarded for reducing the number of “sad face” or related symbols, and praised for the increased number of happy faces or positive symbols, as presented on the chart. Typically the chart is produced by the processing means or a device having processing means associated, e.g. by a communications link, with other processing means processing inputs, objectives and other elements according to embodiments of the invention.

Ideally, a system for developing a subject's voiding awareness and/or assessing a subject's readiness for toilet training, developing the subject's awareness in preparation for toilet training and/or guiding toilet training includes a number of features such as e.g. processing means, communicatively coupled with display means for displaying charts and information, the results of Pattern Tests, Causality Tests, updates on toileting progress, charts and the like, and is also communicatively coupled with input means operable by a user to control operation of the display means and use of the processing means. Typically, a transmitter is provided and configured to transmit signals containing toilet training-related data. Toilet training-related data may be obtained from a sensor associated with an absorbent article worn by the subject. A receiver configured to receive signals from the transmitter is typically provided in connection with the processing means which processes the received signals, and performs Pattern and Causality Tests. The processing means may communicate the outcome of these tests and optionally, e.g. the outcome and/or status of awareness development and related assessments, as well as toilet training information to the display device. This data may also be transmitted to a data clearing house where it may be pooled and used e.g. in the production of demographic data, used for research or commercial planning.

### Objectives 1003

Typically, objectives 1003 utilised according to embodiments of the present invention for establishing or modifying a toileting schedule 1010 may be drawn from the non-exhaustive list provided in Table 1. The preferred outcome (minimised or maximised) of toileting is indicated in UPPERCASE in the columns to the right of each objective. It will be noted that some of the objectives in Table 1 may be regarded as conflicting. For example, objective 4: carer's workload (which affects cost and productivity) is maximised when there is frequent toileting or checking of the subject. However, this conflicts with objective 1: risk of leakage which is minimized when there is frequent toileting/checking. An optimised toileting schedule may not be able to optimise both of these objectives equally. Thus a reduction or hierarchical procedure may be utilized for choosing the best toileting schedule.

The “Risk of Leakage” objective may be improved where actual leakage data is available for a period of observation for a subject (or group of subjects, depending on the scope of the toileting schedule being optimised). In an embodiment, risk of leakage of urine/faecal matter from an incontinence aid may be ascertained for a subject or a group of subjects or indeed for an entire facility by reference to leakage amounts that have been observed (e.g. manually, or estimated using sensors etc.). This may be expressed using linguistic variables such as “large”, “medium” and “small” amounts. Using the actual leakage data, Equation 1 may be applied using three variables “High leakage”, “Moderate Leakage” and “Low Leakage”.

Risk of leakage=α×A+β×B+γ×C Equation 1

A, B and C represent the number of incontinence aids from which there has been a high leakage amount, a moderate leakage amount and a low leakage amount respectively. The coefficients α, β, and γ give a weighting to high, moderate and low leakages with α>β>γ for estimating the worst case scenario for leakage risk. In a more generalized example a leakage may be reported with more variables.

Factors such as previous voided volumes, aid size, time of the day, time elapsed since last event, and/or time elapsed since the aid was applied to the subject may be considered in calculating a toileting schedule 1010 for which an objective 1003 includes minimizing risk of leakage. The risk of leakage probability may be computed according to Equation 2.

$$risk of {leakage}_{updated} \% = risk of {leakage}_{current} \% + f ( voided volume ) * ( 100 \% - risk of {leakage}_{current} \% ) \qquad Equation 2$$

where f( ) may be a linear, S-shape or other function in which the independent and dependent variables are volume and increased probability rate, respectively. The function f( ) may vary for different incontinence aid sizes since smaller aid sizes have higher risk of leakage for the same voided volume.

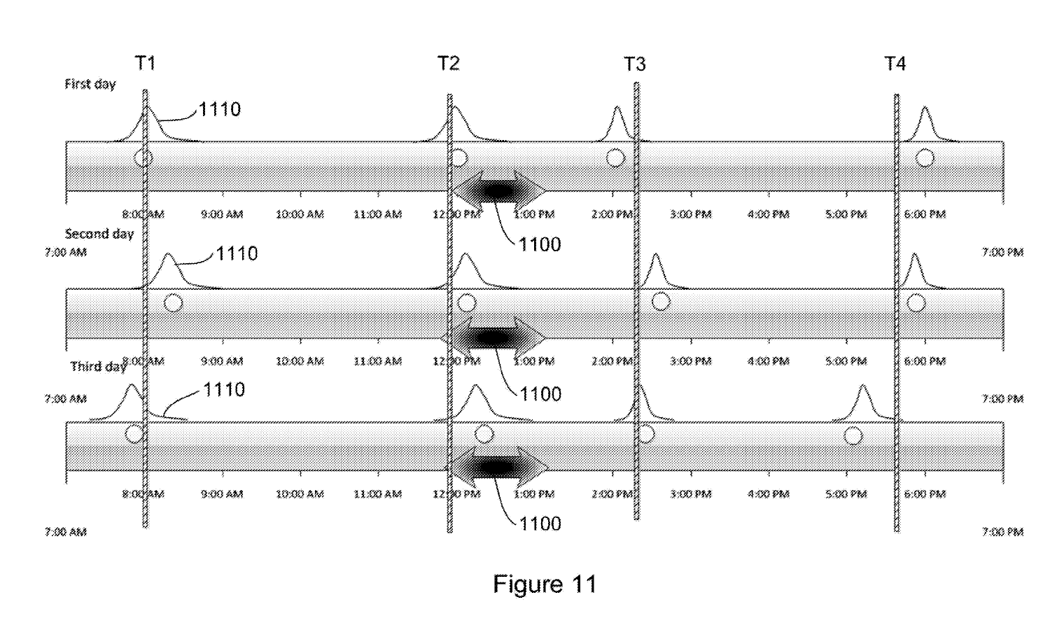

Alternatively/additionally, risk of leakage may increase over time with a linear, S-shape or other function in which the independent and dependent variables are length of time after the last event and/or the duration of time since the aid was applied to the subject and the increase in probability rate, respectively. This probability/risk may be determined according to Equation 3:

$$risk of {leakage}_{updated} = risk of {leakage}_{current} + g ( \begin{matrix}period of time since last event \\ and \text{/} or since a fresh aid was applied\end{matrix} ) * ( 1 - risk of {leakage}_{current} ) \qquad Equation 3$$

where g( ) may be a linear, S-shape or other function. Linear (L), S-shape (S) and square root (SR) functions are represented in FIG. 10, which shows that the increase in risk of leakage probability may approach but never reach 100%. The function g( ) may vary for different times of the day and also different aid sizes, since smaller aid sizes typically have higher risk of leakage for the same voided volume. It is to be understood that the shape of f( ) and g( ) is not limited to the curves shown in FIG. 10.

Equation 4 may be used as an objective function representing objective 2 (Unsuccessful Toileting), which seeks to minimise the volume of urine and/or weight of faecal matter voided into an incontinence aid, rather than into a toilet.

$$unsuccessful toileting = weight of faecal matter in the aid / total weight of dischared feacal + volume of urine in the aid / total volume of discharged urine \qquad Equation 4$$

Alternatively, Equation 5 may be used as an objective function representing objective 2 which seeks to minimise the number of times the subject is taken to the toilet and no voiding occurs:

$$unsuccessful toileting = \frac{ \begin{matrix}number of times a resident is \\ taken to toilet but no voiding happens\end{matrix} }{\begin{matrix}total number of times \\ that a resident is taken to toilet\end{matrix}} \qquad Equation 5$$

In another alternative, an objective function representing objective 2 could be a combination of Equations 4 and 5.

A toileting schedule using minimised unsuccessful toileting as an objective typically provides time indicators for toileting which are close in time and just prior to estimated/expected urinary/faecal events, giving rise to higher probabilities that the event will be voided into a toilet.

Skin problems can occur where there is prolonged wetness or infection. Older skin is thinner, more prone to damage and infection, and typically takes longer to heal when there is a sore or wound. Therefore, minimising skin problems is desirable. Skin problems (Objective 3 in Table 1) may be measured e.g. by monitoring the number of sores arising from lingering moisture in an incontinence aid contacting the subject. Skin problems may be defined by reference to a subjectively or objectively ascertained value. For example, level 1 may designate “healthy” skin; level 2 skin is weaker (e.g. red) level 3 skin is weaker still (e.g. broken), level 4 skin is compromised (e.g. infected), down to a severely compromised skin at level n. A value may be used to represent a level change (LC) in skin quality, over a period of time. For example, if a subject has level 2 skin quality which is downgraded after 7 days to level 5 then LC equates to 2 minus 5 which is minus 3. Negative and positive LC values indicate deterioration and improvement in skin condition over time. Equation 6 may be used to govern overall skin integrity for n subjects being monitored in a care facility:

$$Skin integrity = {\sum }_{s = 1}^{n} {LC}_{s} \qquad Equation 6$$

Factors such as previous voided volumes, aid size, event time-of-day, time elapsed since last event, and/or time elapsed since the aid was applied may be considered in calculating a toileting schedule 1010 for which an objective 1003 includes skin problems, where an objective may be to minimise the duration for which the subject wears a soiled aid. This may also be calculated using a multi-objective procedure employing a combination of the objectives relating e.g. to unsuccessful toileting and risk of leakage.

Carer productivity influences costs. Consequently, it is generally desirable to minimise carer workload associated with toileting subjects so that efficiency and hence carer productivity may be increased. Workload may be established according to e.g. the total number of toileting procedures assisted during a period of time (e.g. a shift), the distance a carer walks to conduct an assisted toileting procedure, time taken to assist a toileting procedure and so on. Minimising variability in workload between carers and between shifts may also be desirable for a facility to ensure fairness and adequate staffing.

The number and size of incontinence aids used for a subject also influences cost of care. It is usually desirable to minimise consumption and hence cost. Aid usage may be measured and optimised in a number of different ways. One example involves an objective function for optimising aid usage provided in Equation 7:

Aid usage=α×A+β×B+γ×C Equation 7

where A, B and C represent the quantity of light, moderate and heavy capacity incontinence aid types consumed, respectively. α, β, and γ are weighting coefficients representing the relative preference in using one pad type against another. Values of α, β, and γ may be varied according to the objectives that are being optimised. For example, in the most simple form the coefficients may each have equal value if only the total number of incontinence aids used is of concern.

If it is preferable to minimise use of heavy capacity incontinence aids (C) then the values are set as γ>β>α. The coefficients may be given an absolute value which may be determined e.g. according to the cost of the various pad types. Thus, where the cost of a heavy, moderate and light capacity pad is $10, $8 and $5 respectively, the values for γ>β>α may be set to 2, 1.6 and 1, or the like. Alternatively, the value of the coefficients may be determined according to a function.

In another embodiment, if the comfort of the subject is another objective 1003 considered, then the values of α, β, and γ may be based on feedback from the subject as to the comfort of using each of the incontinence aid types. This may include different sizes, brands, shapes and the like. For an obese subject, a heavy capacity aid may be more comfortable than a moderate capacity aid, and a moderate capacity aid may be twice as comfortable as a light aid. Thus, values for α, β, and γ may be set to e.g. 1, 2, and 4, respectively. The values of α, β, and γ may be varied for different times of day and other changing circumstances since, for example, the subject might be more comfortable wearing a moderate capacity aid rather than heavy capacity aid at night time, resulting in the value for β being less than the values of α and γ for night time only. Alternatively, a plurality of coefficients may be used to consider multiple factors affecting incontinence aid selection and consumption.

A subject's comfort and dignity (objective 6 in Table 1) can influence social outcomes and quality of life for individuals suffering from incontinence. This objective may be optimised by considering factors such as the number of times the subject is disturbed and/or the subject's incontinence aid is checked unnecessarily, the number of unsuccessful toiletings, the time duration that a subject wears an aid that is ready to be changed, the number of leakages and the like. Interferences to daily life may indicate a non-preferred time to change an aid or toilet the subject e.g. while a subject is sleeping, at meal time or during activity periods such as craft or excursions. These may be referred to as non-preferred times or non-preferred time periods.

Care guidelines may be prescribed by law, mandated, recommended or imposed by a care facility. These guidelines indicate the number of times that a subject should be toileted per day and/or their incontinence aid checked. Objective item 7 enables these guidelines to be considered when establishing a toileting schedule according to embodiments of the present invention.

Subjects may fall due to disorientation, unsteadiness or due to urgency in rushing to a toilet. The latter factor in particular may be mitigated using a toileting schedule which increases the likelihood of adequate time being provided to reach the toilet, and where necessary, adequate support or assistance from a carer. An objective function for addressing the risk of a subject falling (objective 8) may be represented in any suitable way, one of which is in Equation 8:

$$risk of fall for a resident = {\sum }_{i = 1}^{n} {DS}_{i} \qquad Equation 8$$

DSᵢ represents the seriousness of damage or harm resulting from the subject's i^{th} fall when the fall is associated with the subject's incontinence condition. There may be several levels for DS from “no damage” to “serious damage” for which the subject requires significant medical attention.

Incontinence aid leakage has many associated costs such as e.g. energy, water, detergent, labour etc. resulting from attending to leakage events, washing linens, changing sheets and the like. Additional costs may be incurred if there is harm to the subject or damage to infrastructure, compromised hygiene and the like. Environment related costs such as a “waste and environmental levy” may exist. Frequent toileting can lead to more aid changes since carers and subjects may be reluctant to continue wearing the same aid even though it is not soiled to capacity. Infrequent toileting may result in soiled aids which leak. Both scenarios result in heavier waste which may have higher costs. This is considered at objective 9 in Table 1.

### Toileting Schedule 1010

A toileting schedule 1010 is typically generated to indicate when a toileting procedure such as toileting a subject or performing an aid change should occur. A toileting schedule may comprise a set of time indicators which enable identification of a time or a time period at or during which a voiding event is expected to occur and/or a set of time indicators for performing a toileting procedure. The time indicators are optimised by processing means 1002 according to received inputs 1001 and objectives 1003. These time indicators for voiding events and/or toileting procedures are typically used in a care plan for conducting toileting procedures and/or aid changes with a subject.

In a preferred embodiment, a toileting schedule calculated according to the present invention is adapted for display on a display device or printing for a file or bed record. Ideally, the displayed toileting schedule may be configured to show one or both of expected voiding event time indicators and toileting procedure time indicators. Control over the display may be achieved using e.g. a filter option provided via user interface 1400.

A “toileting procedure” is typically used to refer to the work done, from the time the responsible individual (usually a carer) becomes aware that the subject requires toileting, to the time that the subject requires no further assistance in relation to that toileting procedure. Thus, for a for a mobile, otherwise healthy subject with incontinence, a toileting procedure may require only a reminder directed to the subject, whereas for a bed-bound subject a toileting procedure may require significantly more effort, such as moving the subject onto a wheelchair, taking them to toilet where they discharge the bowel/bladder into the toilet and ultimately returning the subject to bed. This toileting procedure may require more than one carer.

Time indicators for expected voiding events may have properties including: (i) ‘genuinity’ which represents how genuine the event occurrence estimate is (low genuinity may correspond to high expectation of a false positive event in the data used to generate the time indicators e.g. due to wet-back, noise, incorrect event detection, data error etc.); (ii) event type which may be e.g. urinary or faecal or mixed; (iii) time indicator exactness representing the likely accuracy of the time indicator for an expected event; (iv) event certainty representing the certainty or confidence that the expected event will occur; (v) event severity representing the seriousness of adverse consequences arising from the event occurring without an appropriate toileting procedure being performed (e.g. if there is no toileting into a toilet and voiding into an incontinence aid occurs).

In an embodiment, an event severity value may be computed based on one or more of (a) anticipated size of the expected event: larger events typically have higher severity value; (b) expected event type: faecal events typically have higher severity value than urinary events; (c) physical and behavioural attributes of the subject (e.g. weight, mobility, mental condition), for example, an expected event of size 100 ml for a mobile subject may have a higher severity value than the same volume of an expected event for a bed-bound subject; (d) time of the day: e.g. the severity value may be lower for events expected to occur at night time as it is preferable that the subject is not disturbed for toileting during sleep. However, if the expected event size is very high, or a faecal event is anticipated, the severity value may be higher irrespective of time of day as in those circumstances the subject should be toileted or checked as close as possible to the time indicator for that event. Any of these scenarios may be treated differently according to the practice of the facility or the carer, requests from relatives and the like which may be represented by inputs 1001 and/or objectives 1003.

Further time indicator properties for expected events may include (vi) certainty of severity representing how certain the system is of the allocated severity for the expected event; (vii) toileting duration representing how long it takes for a carer to conduct a toileting procedure for a subject; and (viii) toileting duration certainty representing how certain the system is in the calculation of toileting duration, to name a few. Properties of carers who may conduct the toileting procedure, and properties of the subject who is toileted may also be incorporated in the schema for a time indicator in a toileting schedule.

FIG. 3 illustrates the types of information that a toileting schedule comprised of time indicators indicative of expected events, may provide. It is to be understood that different data structure/s or representations may be utilised to convey information for a toileting schedule. In an embodiment, each of the genuinity, type, and exactness may be described in more detail by a separate certainty value representing how certain the system is of their values; this is similar to “certainty of severity” as described above. In another embodiment a certainty value itself may be described in more detail by another certainty value defining how certain the certainty value is, and so forth. Multiple levels of confidence/certainty in the information and properties of events in a toileting schedule may be represented in this way.

Properties of a toileting procedure may also be included in the data structure such as e.g. the toileting procedure activity (toileting, pad change, alert to subject, duration of procedure and the like). These properties may be used by the system to determine time indicators for performing toileting procedures. Toileting of the subject (over a commode or receptacle) is scheduled prior to an expected event, whereas an aid change is scheduled after the expected event. The scheduling of the time indicators for toileting procedures may depend on various properties of inputs such as e.g. certainty of data, as well as properties of carers and subjects as discussed below in the examples, particularly in relation to one-to-many, many-to-one and many-to-many carer-to-subject relationships, collision avoidance and the like.

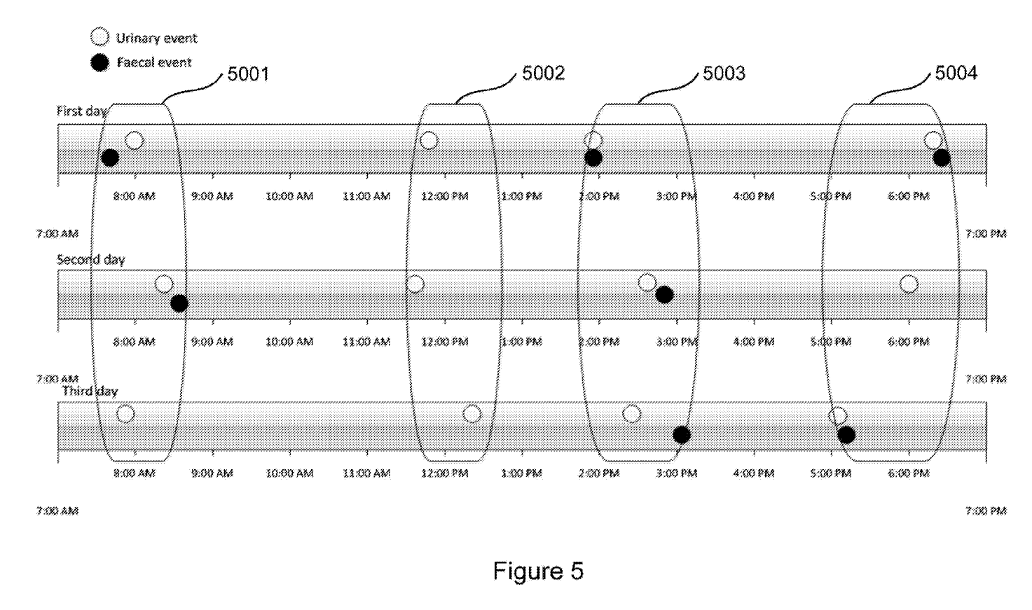

FIG. 4 is an example of toileting schedule for a subject (showing expected voiding events between 7 am to 7 pm) calculated according to an embodiment of the invention. The first voiding event is expected to occur at 11:25 am. This event is expected to be 95% genuine (i.e. there is a predicted 5% chance that there will be no wetness event). “Very high” exactness indicates that the event is expected to occur nearly exactly at 11:25 am. The severity of 15% indicates a relatively low adverse impact if the event is not dealt with by toileting (e.g. there is evacuation into an incontinence aid), particularly given the “high” certainty of the severity value. The second event is expected to occur between 3:30 pm and 4:00 pm. The second event is likely to be a non-genuine event given the “very low” genuinity and exactness of 10%. Also, the severity is reliably small (11% with high certainty). Events of this type (low severity and genuinity) may ignored or given lower priority by carers.

### Example

### Deriving a Toileting Schedule

FIG. 5 shows data for three events sets obtained from a subject between 7 am and 7 pm on three consecutive days labelled First Day, Second Day and Third Day. There are two objectives 1003 for the toileting schedule being derived by the system 1000. These are (1) to minimise risk of leakage and (2) minimize carer's work load. The carer's work load is calculated based on the number of toileting procedures which may be conducted between 7 am and 7 pm. At the conclusion of method 2000 processing means 1002 provides a toileting schedule 1010 identifying the following time indicators which are estimates of the best times for taking the subject to the toilet: 7:30 am to 8:30 am, 11:30 am to 12:40 pm, 2:00 pm to 3:00 pm; and 5:15 pm to 6:30 pm. These are shown in FIG. 5 at 5001, 5002, 5003 and 5004 respectively.

In order to generate a toileting schedule 1010, it is necessary in a step 2004 to express the objectives 1003 received at step 2003 as objective functions 1003a. A mathematical function is used to determine how good a toileting schedule is for a given set of inputs 1001 and objectives 1003. The mathematical function may be determined in numerous different ways, depending e.g. on the type of carer-to-subject relationship. This may include, for example, one carer to one subject (one-to-one), many carers to one subject (many-to-one), one carer to many subjects (one-to-many) and many carers to many subjects (many-to-many) where many designates more than one.

In a one-to-one relationship, a goal of optimisation procedure 2006 may be to optimize the value/s of a mathematical function by seeking the minimum number of toileting procedures necessary to achieve adequate toileting. In another embodiment, a goal of optimisation procedure 2006 may be to align the number of toileting procedures with a property in a “general information” input such as e.g. allowable number of toiletings according to care guidelines. In yet another embodiment a goal may be to optimize one or more of the other objectives given in Table 1. Inputs 1001 may be used directly or indirectly to influence optimization procedure 2006. For example, information pertaining to intakes sets may be used for deriving more detailed information for events sets when events and intakes are linked e.g. by causal relationship. Subject information such as physical characteristics, non-preferred toileting times, continence holding ability, medical conditions and the like may be considered too.

Ideally, optimization procedure 2006 is iterative. For example, expected voiding event. The time indicators in a toileting schedule may be determined by a combination of (i) optimizing (maximizing or minimizing) objective function values, and (ii) applying a function such as a “a relax-period-of-time-function”. A relax-period-of-time-function may receive inputs pertaining to an expected event (event A) with one or more associated properties and then calculate a time indicator for the occurrence of the next expected event (event B) with its associated properties. Then a time indicator and properties for the next expected event in time (event C) can be calculated by applying the relax-period-of-time-function on event B and so forth.

If data used in optimization procedure 2006 contains sparse periods, in some embodiments time indicators and associated expected events and their properties in a toileting schedule may still be calculated by applying a relax-period-of-time-function. Alternatively/additionally, time indicators may be calculated (e.g. with less certainty) using additional input information pertaining e.g. to physical and/or medical condition/s of the subject (such as digestive behaviour) and other general information which enable the system to generate a “best estimate” of a time indicator for an expected event. These time indicators can then be used to identify time indicators for performing a toileting procedure despite the sparse period of data used to calculate it.

The objective functions 1003a may be utilized by applying a function such as a one-to-one distance function which measures the sum of the distances of each time indicator to its nearest actual event. Alternatively, the distances of each time indicator to its nearest estimated event may be aggregated with other input information by a suitable aggregate function.

In an embodiment, a one-to-one distance function is based on one or more of received input data 1001 (such as one or more of the properties of void events, subjects, and/or general information, the subject's bladder/bowel holding ability), estimated event size, certainty of occurrence, certainty of event size, and expected event type to name a few. For example, a one-to-one distance function may be represented as:

CoO*ES*CoES*(D)*(1−PoHA) Equation 9

or

CoO*ES*CoES*(D) Equation 10

where: CoO is certainty of expected event occurring;
ES is the expected event size;
CoES is the certainty of expected event size;
D is the distance from the time indicator of the actual event to the closest chosen time indicator selected, or in the process of being selected, by the optimisation procedure;
PoHA is probability of holding computed from subject holding time.

Different variations for the one-to-one distance function with different operations and/or variables may be generated similarly. A general one-to-one objective function may take the form:

f(PoID,PiT) Equation 11

where: PoID represents properties of the input data;
PiT represents properties of expected event time indicators (points in time); and
the output of f is of ordinary type.

Events which are unlikely to be captured by any of the expected event time indicators in a toileting schedule may be identified as non-captured events. A non-captured event objective function may deal with non-captured events and may be determined based on the subject's continence holding ability and the distances between non-captured events and their nearest time indicator/s calculated for expected event/s.

In an embodiment, the one-to-one distance function and the non-captured event objective function are utilized by a multi-objective optimization procedure. The optimization procedure may “fail” or disallow a toileting schedule or parts of it if one or more actual events are not captured, unless failure to capture an event is consequential to a non-preferred time input received in step 2001.

Non-preferred time inputs representing times when toileting procedures should be avoided may be based on properties of a subject, carer or general information and may be dealt with using a non-preference objective function. Alternatively, the optimization procedure may disallow scheduling of a toileting event during a non-preferred time. The value of a non-preference objective function may be determined based on input data, and/or a degree of non-preference input, time of day, physical characteristic (e.g. weight, mobility ant etc.), medical condition and the like.

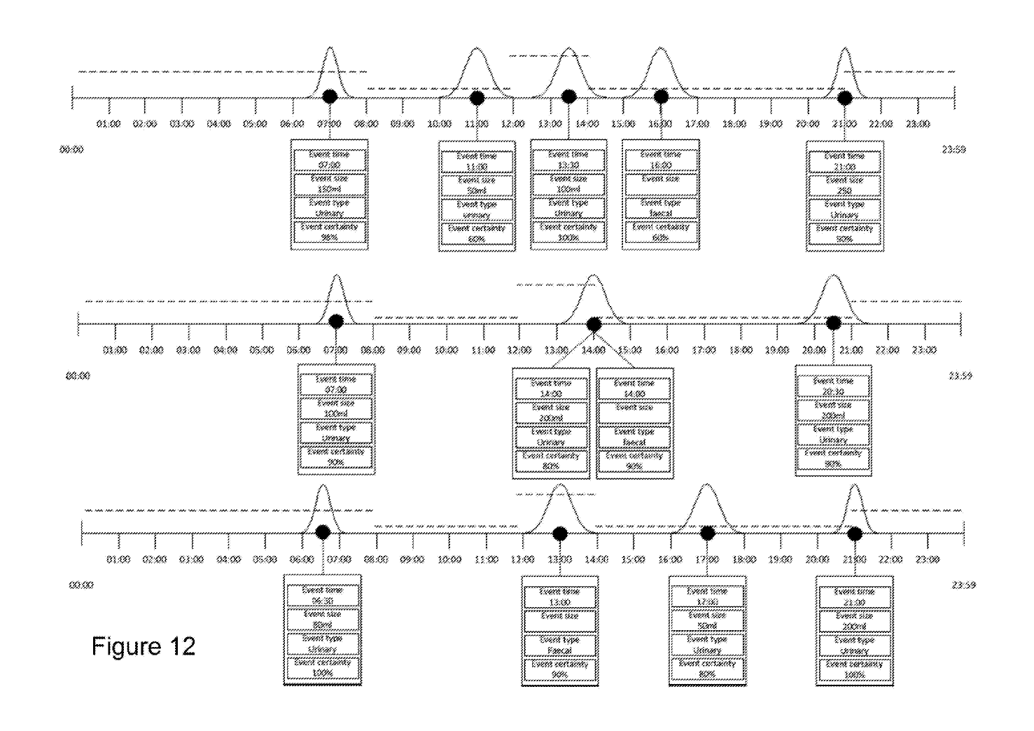

FIG. 11 illustrates schematically how non-preferred toileting times may be considered in deriving a toileting profile. The double side arrows 1100, show a non-preferred periods of time with the darker areas indicating a time period with higher degree of non-preference. Time indicators for expected voiding events in the schedule are represented by probability distributions 1110. The first calculated toileting procedure is designated at T1, the second at T2, the third at T3 and the fourth at T4. Due to the non-preferred period of time, second toileting procedure time T2 has been scheduled at 12:00 pm, although if the non-preferred period of time did not apply the probability distributions 1110 have T2 at scheduled about 12:15 pm. The profile illustrated in FIG. 11 is just one example of how an optimization method, according to embodiments of the invention, may be brought into effect utilising different procedures, inputs, objectives and the like. A further alternative may compute a non-preference value as:

D*PoDP Equation 12

where D is the distance from the time indicator of the estimated event to a chosen point in time and PoDP is the probability of the chosen point coinciding with a “non-preferred”period of time. In this embodiment, the toileting schedule may be discarded if it contains time indicators where:
PoDP is greater than a threshold; and/or
the number of time indicators in a non-preference period exceeds an acceptable threshold; and/or
a threshold number of time indicators in a period of time have an associated PoDP which exceeds a threshold.
   The thresholds may be set manually, based on inputs or determined according to a function.

### Example

### One-to-One Toileting Schedule

In an embodiment, a “one-to-one-objective-function” may be derived by employing a multi-objective procedure using a plurality of objective functions influencing an optimal time for a toileting procedure for a subject. These may include a one to one distance function, non-captured event objective function and non-preferred time objective function. A one to one distance function and non-captured event objective function may be used for measuring objectives such as risk of leakage, unsuccessful toileting, skin integrity, aid usage and the like as discussed with reference to the objectives in Table 1. The non-preferred time objective function may be used to capture objectives such as subject comfort, carer availability, work load also discussed to an extent in Table 1. The objectives in Table 1 or any other objectives may be implemented in any manner, such as the approaches discussed herein under the heading “Objectives 1003”.

Alternatively, objective functions may be dealt with separately. Here, different possible toileting schedules are compared by processing means 1002 which is configurable to identify a toileting schedule that is best able to satisfy all applicable objectives. Alternatively, toileting schedules may be identified as “Pareto-Equivalent”, and the multi-objective procedure may be applied until the Pareto-Equivalency is eliminated. Two or more toileting schedules are considered Pareto Equivalent if i) none of the toileting schedules outperform the other toileting schedules in all the objective functions; and 2) none of the objective functions can be improved in value without diminishing some other objective function's value. Without additional preference information, all Pareto Equivalent toileting schedules can be considered equally good. Manual intervention may also be permitted to eliminate Pareto-Equivalency, by manual input to system 1000 e.g. via a user interface 1400.

In an embodiment, an objective reduction procedure may choose n objective functions from the total set of m objective functions, where m≧n. The chosen n objective functions are then combined to form r objective functions (where m≧r≧0). In the case of r=n, the number of the objective functions remains the same but they are combined to form a different set of functions. In the case of r=1 all n original objectives are combined to form only one objective function. As an example: objective functions A, B, and C can form 2 new objective functions, N1 and N2, as follows; N1=f(A+B,C), N2=g(N1+C), where f and g are functions. An objective reduction procedure may use a weighting scheme, in which one or more of the objectives are weighted based on their importance, and then combined.

A combination procedure utilizes the objectives 1003 to form c objective functions 1003a (where c≦m). As an example: objectives A, B, and C can form 3 new objective functions, N1 and N2, N3, as follows; N1=f(A+B), N2=g(N1+C), N3=(A,B,C), where f and g are functions. An objective combination procedure may use a weighting scheme, in which one or more of the objective functions are weighted based on their relative importance, and then combined.

Alternatively/additionally, a hierarchical ranking procedure may be performed to compare the value of objective functions of two or more toileting schedules according to their ranking (order of importance). Here, the values of the same objectives with the highest ranking from one or more toileting schedules are compared and the toileting schedule with the best objective value is selected as the optimized toileting schedule. For example if there are two objectives A and B (with A being more important than B) then toileting schedule T1 is said to be better than toileting schedule T2 if value of objective A in T1 is better than the value of objective A in T2. If the value of objective A is the same for both T1 and T2 then the value of objective B is considered for deciding which toileting schedule is better. In another embodiment, the objective functions may be sorted based on their ranking and compared between one or more toileting schedules.

The best objective function in each toileting schedule increases the “goodness” of that particular toileting schedule. The increase in the goodness is typically computed based on one or more of the ranking (relative importance) and objective value/s. In this approach the values of the objective functions are weighted, e.g. the system calculates for each of T1 and T2 the value of objective A plus ten times the value of objective B, and then compares the results for both T1 and T2 to define which is better. In yet another embodiment where the values of the highest ranked objectives in one or more toileting schedules are close to each other, e.g. within ±1, the values of the second highest ranked objective of the one or more toileting schedules are compared. If those values are also close to each other, the comparison continues with value of the next highest ranking objective value until one toileting schedule is identified as superior in terms of its objective when compared to the other toileting schedules. Scenarios 1 to 3 demonstrate one application of the inventive methodology to calculating a toileting schedule for a one-to-one relationship.

### Example

### One-to-Many Toileting Schedule

In a one-to-many relationship (one carer to many subjects), goals may include goals of a one-to-one relationship, as well as optimizing values of the objectives received in respect of individual subjects in the relationship, optimizing a sum of the values of objectives for individual subjects, and optimizing the productivity of the carer. A toileting schedule for each subject in a one-to-many relationship may be derived in a manner similar to that described for a one-to-one relationship, with an additional one-to-many adjustment step. This may involve using inputs 1001 to seek time indicators for toileting a particular subject while a particular one-to-many distance function is minimized. The one-to-many distance function may be the same as the one-to-one objective function, with a further adjustment step to deal with potential collision of scheduled toileting times for a plurality of subjects. This may be referred to as a one-to-many collision avoidance objective function.

In an embodiment, the one-to-many collision avoidance objective function causes processing means 1002 to disallow a toileting schedule (or part thereof), where there is overlap between time indicators for conducting a toileting procedure for different subjects by one carer. The “one-to-one objective function” and collision avoidance objective function may be utilized by a multi-objective procedure for applying objective functions 1003a. The value of the one-to-many collision avoidance function may be determined based on inputs including but not limited to: expected voiding event time indicators, estimated duration of toileting procedures (and certainty thereof), required carer support time, performance of the carer and so on. For example if processing means 1002 calculates a toileting schedule 1010 scheduling: (i) a an expected voiding event for subject A at 11:20 am and subject A has a continence holding ability of ±20 mins, and (ii) an expected voiding event for subject B at 11:30 am and subject B has no continence holding ability; and (iii) the support time for both A and B is 10 minutes; then the one-to-many collision avoidance objective function schedules a toileting procedure for subject B first and then subject A.

A one-to-many objective function may be derived by employing a multi-objective procedure on the one-to-many collision avoidance function, and/or a “one-to-one objective function”.

In an embodiment, the optimum number of subjects that a carer can support while meeting the objectives captured by the one-to-many objective function may be determined in an iterative process by decrementing the total number of subjects (or incrementing from a low initial number of subjects) and then applying the one-to-many procedure until an optimised value of the one-to-many objective function is reached.

### Example

### Many-to-One Toileting Schedule

In a many-to-one relationship (many carers to one subject), goals may include goals of a one-to-one relationship, as well as optimizing values of objectives received for the subject, and/or carers in the relationship e.g. to optimize a sum of the values of objectives for the carers as a group (e.g. productivity) and/or values of objectives (such as productivity) for individual carers. A toileting schedule for a subject in a many-to-one relationship may be derived in a manner similar to that described for a one-to-one relationship, with an additional many-to-one adjustment step. The many-to-one objective function may be similar to a one-to-one objective function, with one or more adjustment steps, i.e. additional objectives incorporated into the function to deal with collision of carers, to avoid assigning more carers than necessary to an event in the schedule; and workload distribution across a shift (e.g. carer A is assigned one event in the morning and one in the afternoon with the same total event duration); and workload distribution among carers (e.g. Carers A, B, and C have comparable workloads of 45, 49, and 47 minutes per day respectively),

If the value of a many-to-one collision objective function is high (indicating too many carers attending an event in a schedule) or a workload distribution objective function is high (indicating uneven workload for a carer shift or between carers) then the processing means 1002 may “fail” the toileting schedule or parts of it. A many-to-one collision avoidance function and many-to-one workload distribution function may be determined by processing means 1002 based on inputs 1001 such as e.g. expected voiding event (voiding) time and/or (and certainty thereof), toileting procedure time and/or duration, carer productivity, carer location, subject location, toilet location and the like. These maybe determined directly from the inputs, or derived from or with reference to the literature or manual inputs received by processing means 1002 e.g. by user interface 1400. Alternatively, a multi-objective procedure may be utilized with the many-to-one collision avoidance objective function, workload distribution objective function, and a “one-to-one objective function” to form the objective functions 1003a.

The optimum number of carers required to optimise the many-to-one objective function may be determined in an iterative process e.g. by decrementing the available number of carers (or incrementing from a low initial number of carers) and then applying the many-to-one procedure until a desired (optimised) value of the many-to-one objective function is reached.

### Example

### Many-to-Many Toileting Schedule

In a many-to-many relationship (many carers to many subjects), goals may include goals of a one-to-many relationship, and goals of a many-to-one relationship. A toileting schedule for each subject in a many-to-many relationship may be derived in a manner similar to that described for a one-to-many or many-to-one relationship, with one or both of the many-to-one or one-to-many adjustment steps. Toileting schedules may also be derived from the perspective of one or more carers in a many-to-many relationship, using similar techniques.

Inputs 1001 may be used to seek time indicators, and the identity of one or more carers and the identity of a subject needing to undergo a toileting procedure by e.g. seeking to minimise the value of a chosen many-to-many distance function. The many-to-many distance function may comprise one or more of the one-to-many and many-to-one distance functions as described above. As is the case in all embodiments, sparse time periods, non-preference time periods, workloads and collision avoidance objectives may be accounted for in the toileting schedule using one or more of the special functions and techniques described above. These functions may be combined, or employed separately in sequence, typically in a hierarchal order. In an embodiment a many-to-many objective function is derived by applying multi-objective optimization on many-to-one and one-to-many objective functions.

The number of carers required to optimise a many-to-many objective function may be computed by incrementing an initial number of carers (or decrementing from a maximum number of available carers) until a desired (optimised) value of the many-to-many objective function is reached. The number of subjects for an optimal many to many relationship may be computed in a similar manner.

Scenarios 4 to 6 demonstrate application of the inventive methodology to calculate a toileting schedule for a many-to-many relationship.

In various embodiments, properties such as expected voiding event duration (and certainty thereof), toileting procedure duration, reported productivity of the carer and the like may be determined according to data received as inputs 1001, by reference to literature, or they may be defined manually. In another embodiment toileting procedure duration (and certainty thereof) may be calculated based on one or more other properties such as physical characteristics of a subject (e.g. weight, medical condition, mobility, PEG fed, etc.), carer performance, carer location, subject location, bathroom/toilet location and the like.

Optimisation procedures employed in execution of embodiments of the invention may have many different characteristics, some of which are discussed below with reference to parallel, multi-level and hybrid optimisation procedures.

### Parallel Optimization

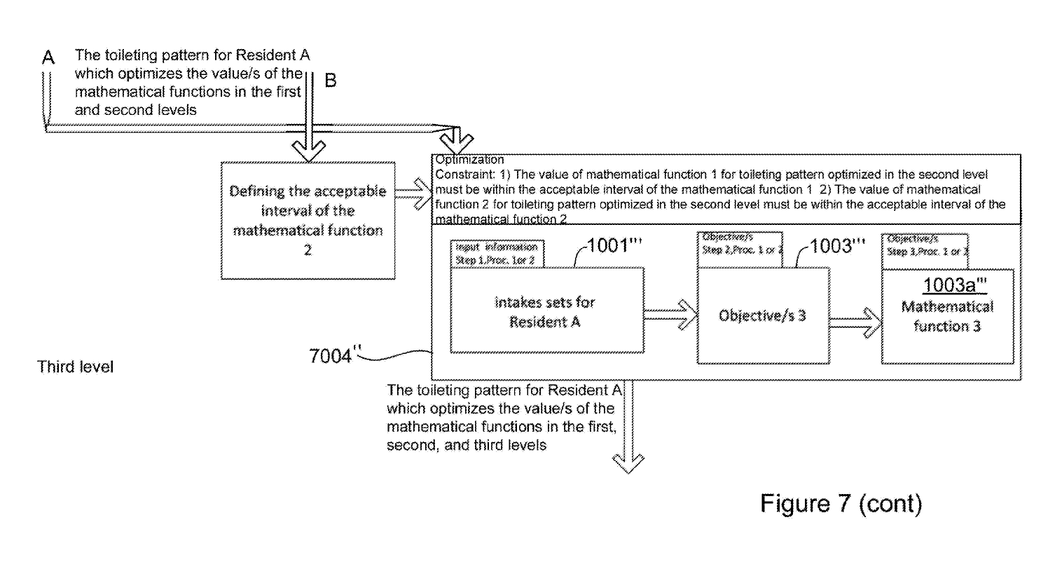

FIG. 6 presents a “parallel” optimization procedure 6000 showing the specific inputs 6001: intake set, event set and carer availability for a subject referred to as Resident A. The objective/s 6003 are shown generically (i.e. specific objectives are not defined in FIG. 6). Parallel optimisation is typically suitable for cases where the relative importance, or a combination of ranked and relative importance of the objectives 6003 can be determined. An example of an objective function 1003a utilising a reduction procedure executed with three objectives: O₁, O₂ and O₃ each having importance defined as: W₁, W₂ and W₃ respectively, may be defined mathematically according to Equation 13:

$$result of objective reduction procedure = {\sum }_{i = 1}^{3} {W}_{i} {O}_{i} \qquad Equation 13$$

### Multi-Level Optimisation

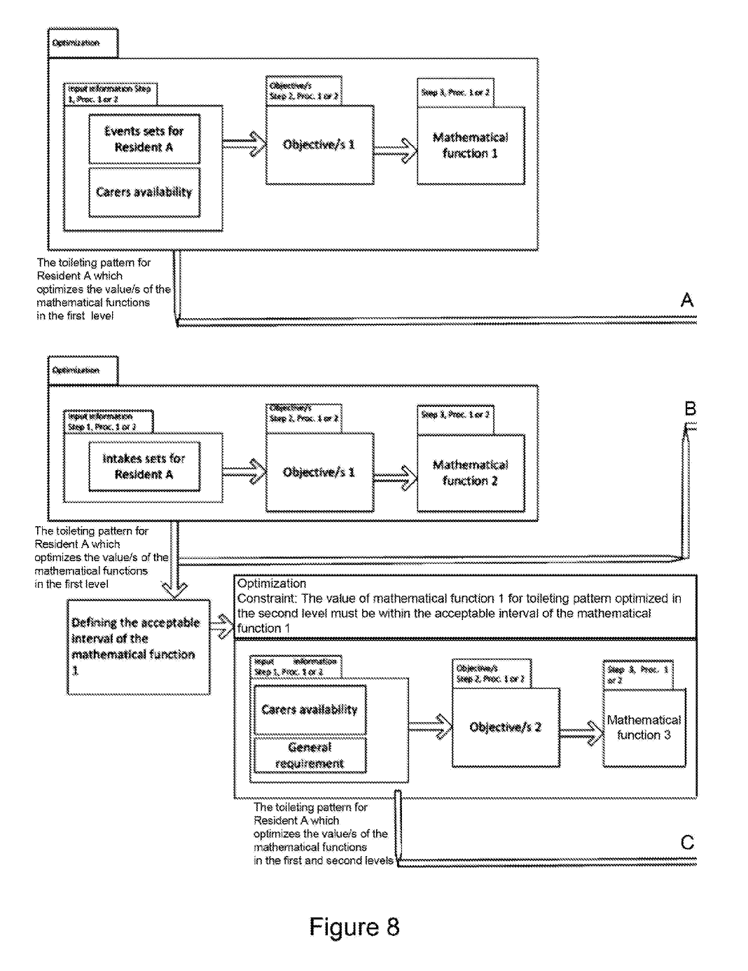

Multiple iterations of optimization procedures may be employed in a multi-level optimisation procedure 7000. In each level, a toileting schedule 1010 which optimizes the values of objective functions 1003a for the received inputs 1001 is determined by processing means 1002. Values extracted from a mathematical function corresponding to the optimized toileting schedule may have one or more acceptable intervals 7004. For example, if the calculated value of the mathematical function for the optimized toileting schedule is x, then the acceptable interval may be [x−α,x+β] (where α and β≦0, may be arbitrarily selected or determined according to some other function). In each subsequent level of the optimization procedure, the processing means solves the mathematical functions to determine an updated toileting schedule 1010 which optimises the value of the mathematical function at that optimisation level, while maintaining the value of the mathematical function in other levels of the optimisation within the acceptable interval from previous level (or previous levels). The acceptable interval for the next optimisation level is shown as 7004. An example of a multi-level optimisation is illustrated in FIG. 7 for a one-to-one subject-to-carer relationship. One-to-many, many-to-one, and many-to-many subject-carer relationships can be treated similarly.

Multi-level optimisation may suit calculating toileting schedules 1010 using ranked objectives or a combination of ranked and relative importance objectives. In multi-level optimisation, more important objectives are placed in higher (earlier) levels of the multi-level procedure. Thus, the acceptable intervals 7004 for those objective functions influence (carry over) into all other subsequent levels of the multi-level optimisation procedure. One or more objectives may be used in a single optimisation level if they have same (or similar) importance/ranking. For example if three objectives O₁, O₂ and O₃ are ranked as first, first and second respectively, then O₁, O₂ may be used in the first level and O₃ in the second level. The objectives in the first level in this example may be treated by an objective reduction procedure or hierarchical procedure as discussed above.

Inputs 1001 may be applied to the optimisation at any level independently of inputs applied in other levels. Depending on the goal/s of the toileting schedule, the impact of inputs may be influenced e.g. by ranking or weighting the inputs so that some input information carries greater influence in the optimization procedure, particularly in top (i.e. initial) levels of a multi-level optimization procedure.

### Hybrid Optimisation

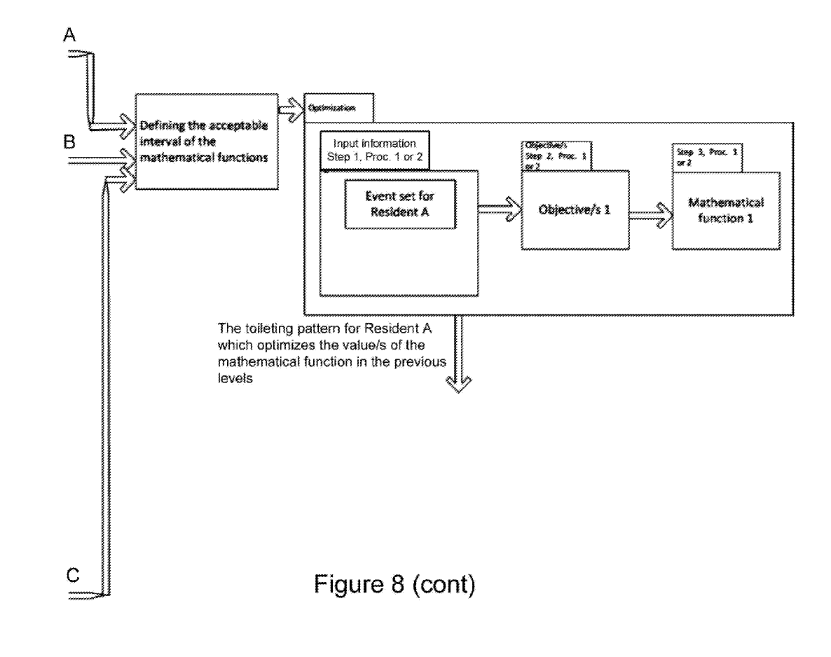

Hybrid optimisation involves a combination of parallel and multi-level optimization procedures described above. FIG. 8 is an example of hybrid procedure for a one-to-one subject-to-carer relationship. One-to-many, many-to-one, and many-to-many relationships can be treated similarly.

Hybrid optimisation may be particularly suitable for cases where a combination of ranked and relative importance is available for objectives 1003. For example, objectives: O₁, O₂ and O₃ are ranked as first, second and third respectively whereas objectives: and O₄, and O₅ are ranked as fourth and fifth respectively with O₄ having three times the importance of O₅. A multi-level procedure may be applied for objectives O₁, O₂ and O₃. The values calculated for their objective functions 1003a can be used as constraints on a parallel procedure with O₄ (with multiplier), and O₅ being treated together using a reduction procedure.

### Example

### Scenario 1

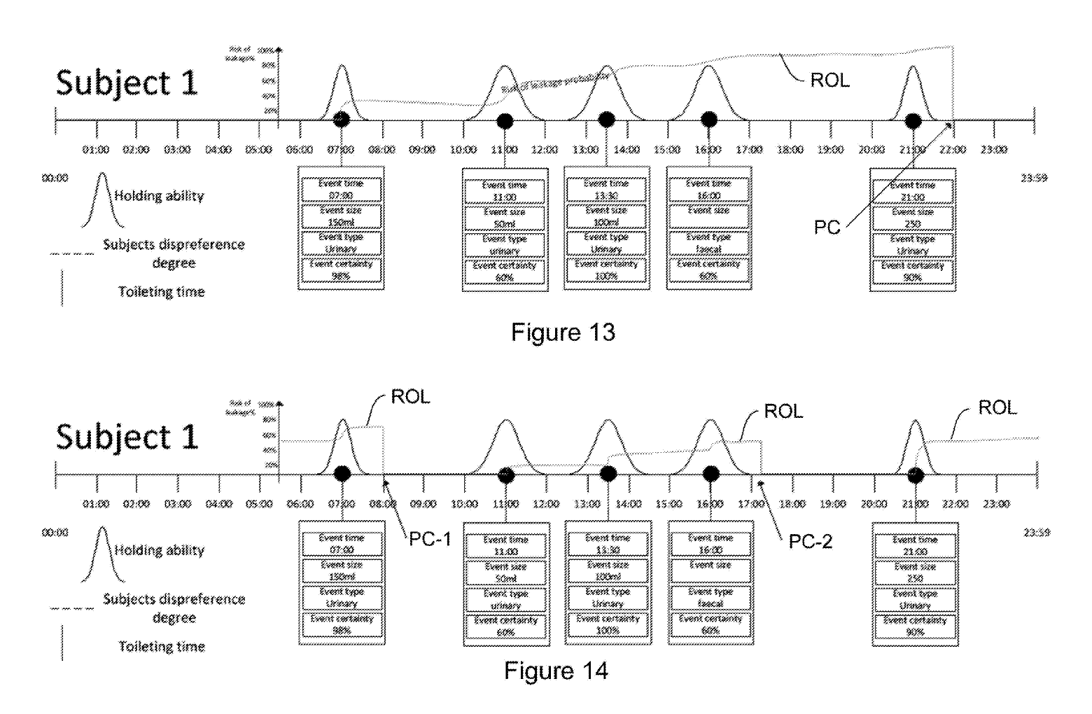

FIG. 12 illustrates graphically, information presented in Table 4 consisting of inputs 1001 for a one-to-one carer-to-subject relationship. Carer availability is given in a time frame, in 24 hour format. For degree of availability, zero indicates “unavailable” whereas 1 indicates available. Event information comprises observed event information received as an input 1001.

A subject's continence holding ability is recorded as a duration of time that the subject can “hold on” without evacuating the bowel or bladder despite the readiness to do so, and may be implemented using a probability distribution with μ=event time indicator and σ2=absolute value of continence holding ability. Similarly, non-preference degree may be represented by a uniform distribution. However, it is to be understood that for these properties and others, other types of distributions such as uniform distribution binomial distribution, half Gaussian distribution etc. may be suitable.

TABLE 5

| Input | | |
|---|---|---|
| category | Schema | Input properties |
| Carer | (time window | (00:01 to 08:00, 0.5), (08:01 to |
| information | available, Degree of | 12:00, 1.0), (12:01 to 14:00, 0.8), |
| | availability) | (14.01 to 24:00, 1) |
| Event | (event time, event | Day 1 |
| information | size, event type, | (07:00, 150 ml, urinary, 98%), |
| | event certainty) | (11:00, 50 ml, urinary, 60%), |
| | | (13:30, 100 ml, urinary, 100%) |
| | | (16:00, faecal, 60%) |
| | | (21:00, 250 ml, urinary, 90%) |
| | | Day 2 |
| | | (07:00, 100 ml, urinary, 90%), |
| | | (14:00, 200 ml, urinary, 80%) |
| | | (14:00, faecal, 90%) |
| | | (20:30, 200 ml, urinary, 90%) |
| | | Day 3 |
| | | (06:30, 80 ml, urinary, 100%), |
| | | (13:00, faecal, 90%) |
| | | (17:00, 50 ml, urinary, 80%) |
| | | (21:00, 200 ml, urinary, 100%) |
| Subject | (event time indicator, | (00:00 to 08:00, 30 mins, ±10 mins), |
| information | support duration, | (08:00 to 21:00, 20 mins, ±20 mins) |
| | continence holding | 20:00 to 24:00, 30 mins, ±10 mins) |
| | ability) | (21:00 to 08:00, 50%) sleeping |
| | (non-preferred time, | (08:00 to 12:00, 10%) |
| | non-preferred degree) | (12:00 to 14:00, 90%) lunch |
| | | (14:00 to 21:00, 10%) |
| General | Preferred number of | 3 changes = highly preferred |
| information | changes per day | 4 changes = moderately preferred |
| | | 1, 2, 5, 6+ changes = not preferred |

For Example 1, processing means 1002 receives objectives 1003 identified in Table 6 with relative importance as indicated, for the purpose of deriving a toileting schedule based on inputs 1001 listed in Table 5.

TABLE 6

| Objective | Comparative importance |
|---|---|
| Successful toileting | 4 |
| Subject comfort (preferred times) | 3 |
| Compliance with regulations | 1 |

Table 7 identifies the control variables used in determining the toileting schedule.

TABLE 7

| Mathematical | |
|---|---|
| description | Long description of control variable |
| E_{s, i} | The i^{th} event of s^{th} subject |
| TTS_{s, j} | The j^{th} time indicator of s^{th} subject |
| NTS | Nearest toileting time indicator to event being examined |
| PNC_{s, i} = | Probability of not capturing E_{s, i} for a given NTS |
| 2 ∫_{NTS}^{NPTS} D_{s, i}(t)dt | |
| NPTS | The nearest peak time indicator to a given NTS |
| D_{s, i}(t) | The continence holding ability distribution curve for an |
| | event like E_{s, i} |
| DT_{s, j} | Discomfort of toileting for Subject S during the j^{th} time |
| | indicator |

PNCs,i may be determined by computing the area under a distribution curve representing the subject's continence holding ability for the event, which is referred to as Ds,i(t) from NTS to the reflection of the peak of the distribution curve on the time axis, referred to as PTS. PTS may be a single point, a set of points, an interval, or a set of intervals depending on the type of distribution curve. In the case of a plurality of peak points, the nearest peak point to NTS is chosen, and is referred to as Nearest Peak Time Indicator, NPTS.

PNCs,i may be determined according to the equation shown in Table 7. Alternatively, if continence holding ability is not known, the Euclidian distance between the NTS and the time indicator of event Es,i may be used instead. In a further alternative, PNCs,i may be calculated as a weighted probability where different types of events (e.g. urinary versus faecal) have different importance. For instance, if it is twice as important to capture a faecal event as it is to capture a urinary event, a weighting coefficient w may be utilised where w is set to 2 for faecal events and 1 for urinary events, such that the weighted probability of not capturing event E_{s,i} can be calculated according to Equation 13:

Weighted Probability of not capturing Eᵢ=2w∫_{NTS}^{NPTS}D_{s,i}(t)dt Equation 13

Typically, it is preferable to capture larger size events. Where the size of expected events can be estimated from the inputs, then a function f(Size of E_{s,i}) can be used to determine the importance of capturing a particular event E_{s,i}. Function f may have sigmoid, linear, constant, root square, exponential or any other characteristic. A sigmoid function will give similar weighting to small expected events and large expected events; whereas an exponential function gives higher weightings for higher volume expected events. In this example a constant weighting is applied, giving the same weighting to all the expected event sizes as in Equation 14:

f(Size of E_{s,i})=(constant)·(Size of E_{s,i}) Equation 14

Also, it is generally preferable to schedule toileting procedures, in the creation of a toileting schedule, for expected events that have higher certainty. A value or a function for determining a certainty value such as g(certainty for E_{s,i}) may be used to ensure expected events having higher certainty are given some precedence. Function g may have sigmoid, linear, constant, root square, exponential or any other characteristic. A sigmoid function will give similar weighting to expected events with low and high certainty; an exponential function gives higher weightings for higher certainty expected events. In this example, a constant which gives same weighting to all certainties is given in Equation 15:

g(certainty of E_{s,i})=(constant)·(certainty of E_{s,i}) Equation 15

Equation 13 may be modified to take account of the certainty and size of expected events, as in Equation 16, which may use one or more of w, f, and g depending on their availability as inputs 1001:

goodness of capturing event i=2wf(Size of E_{s,i})g(certainty of E_{s,i})∫_{NTS}^{NPTS}D_{s,i}(t)dt Equation 16

The goodness of capturing all the events for the subject s may be computed using Equation 17.

$$Goodness of capturing all the events of subject s = 2 {\sum }_{i = 1}^{n} wf ( Size of {E}_{s , i} ) g ( certainty of {E}_{s , i} ) {\int }_{NTS}^{NPTS} {D}_{s , i} ( t ) d t \qquad Equation 17$$

A non-preferred time period for time indicators in the toileting schedule may be ascertained according to the discomfort caused to a subject when toileted or (aid checked) at certain times and according to non-preference degrees. DT_{s,j} may be defined as the discomfort of toileting at TTS_{s,j}, where j is the index of the toileting events and s is the index of the subject DT_{s,j} may be measured by non-preference degree (DD_{s,j}) at TTS_{s,j}. Non-preferred time/s may be determined e.g. by summing all the non-preference values of a given schedule with m time indicators as given in Equation 18.

$$Non \text{-} preferred times = \frac{{\sum }_{j = 1}^{m} {DT}_{s , j}}{m} \qquad Equation 18$$

The total number of toileting events planned by the toileting schedule for a time period may be represented by a value determined according to Equation 19.

$$No . of Events = \{ \begin{matrix}0 , & if number of toileting equals 3 \\ 0.5 , & if number of toileting equals 4 \\ 1 , & if number of toileting not equals 3 or 4\end{matrix} \qquad Equation 19$$

If a parallel optimization procedure is employed then all the received objectives 1003 may be combined according to their relative importance. Objective values computed from Equations 17 and 18 may be normalized first. For normalization one may compute the minimum and maximum values of Equations 17 and 18 with the same number of toiletings and assign a normalization function which maps the maximum value to one and the minimum value to zero. Linear, sigmoid or other types of normalization functions may be used. For example if maximum and minimum values of the objective function given in Equation 17 with 3 toileting events are maximum value and minimum value then normalization function, N( ) has the following properties:
N(minimum value)=0
N(maximum value)=1
N is an increasing function
   Alternatively N( ) may be a decreasing function in which N(minimum value)=1 N(maximum value)=0 and the optimization problem becomes a maximization problem.

Minimization in a parallel procedure may produce the total objective function given in Equation 20 below. Note that the value of Equation 20 does not require normalization as it is already in its normalized form.

$$total objective function = 4 * N ( Goodness of capturing all the events of subjects ) + 3 * N ( Non \text{-} preferred times Error ! Reference source not found . ) + 1 * Number of Events \qquad Equation 20$$

If there is more than one full day of input information, the most recent input information may be given greater influence by applying a higher weighting to inputs corresponding to more recent events in Equation 17.

### Example

### Scenario 2

In the Scenario 2 example, the inputs are identical to those of the Scenario 1 example however the objectives are ranked differently, as shown in Table 8. The objective functions remain as defined in Equations 17, 18 and 19. A multi-level procedure may be employed, wherein the first level solution minimizes the value of the “Goodness of capturing all the events of subject s” objective function (Equation 17). This value, level1ₘᵢₙ is then used as a constraint in the second level. The second level solution minimizes the value of the non-preferred times objective function (Equation 18), the value of which must be less than level1ₘᵢₙ+margin1. The minimum value of the second level: level2ₘᵢₙ together with the value from level 1: level1ₘᵢₙ are then used as constraints in the third level of optimisation. The third level solution optimises (typically this means minimizes) the Number of Events (Equation 19). The values of Equation 17 and Equation 18 for the optimisation being calculated must be smaller than level1ₘᵢₙ+margin1′ and level2ₘᵢₙ+margin2, respectively. The margins represent the intervals α and β discussed under the heading “Multi-level Optimisation”. They may be obtained as a portion of minimum values or some other function, may be a constant e.g. determined by trial and error or some other value.

TABLE 8

| Objective function | Rank |
|---|---|
| the Goodness of capturing all the events of the subject | 1 |
| Non-preferred times | 2 |
| Number of events | 3 |

### Example

### Scenario 3

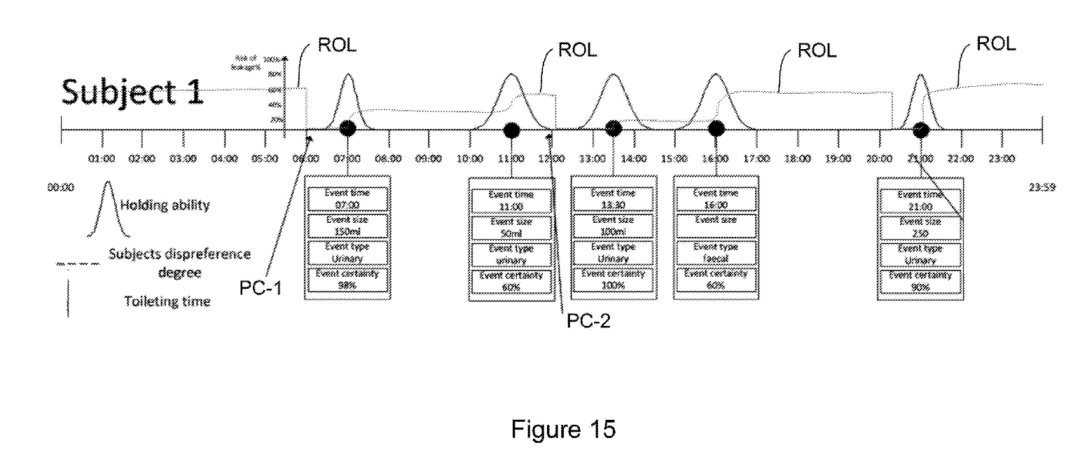

FIG. 13 represents observation data for Subject 1 received as inputs 1001 to processing means 1002; For Scenario 3, the objective 1003 is to derive a toileting schedule 1010 in which the risk of leakage from an incontinence aid worn by Subject 1 remains below 60%. An example of a procedure for determining a toileting schedule for Subject 1 is as follows:

(Scenario 3 Procedure)

| | |
|---|---|
| {step 1} | n= 1 //(where n is no. of aid changes) |

| | |
|---|---|
| {step 2} | risk of leakage is below 60%= false |

| | |
|---|---|
| {step 3} | WHILE (risk of leakage is below 60%= false) |

| | |
|---|---|
| {step 4} | Toileting Schedule= compute the n time indicators that |
| | minimize risk of leakage |

| | |
|---|---|
| {step 5} | Risk of Leakage Percentage= risk of leakage for |
| | current Toileting Schedule |
| {step 6} | IF (Risk of Leakage Percentage < 60%) |

Risk of leakage is below 60%= true

| | |
|---|---|
| {step 7} | ELSE: |

n=n+1

ENDENDWHILE

Parallel optimisation may be used for the Scenario 3 Procedure since there is only one objective.

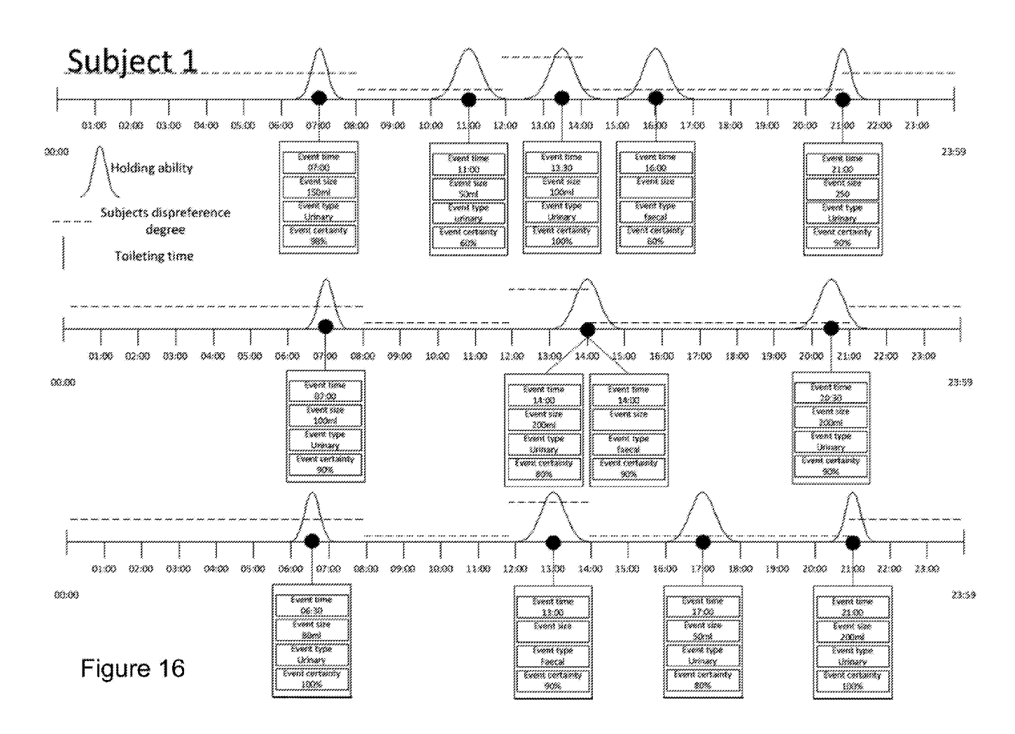

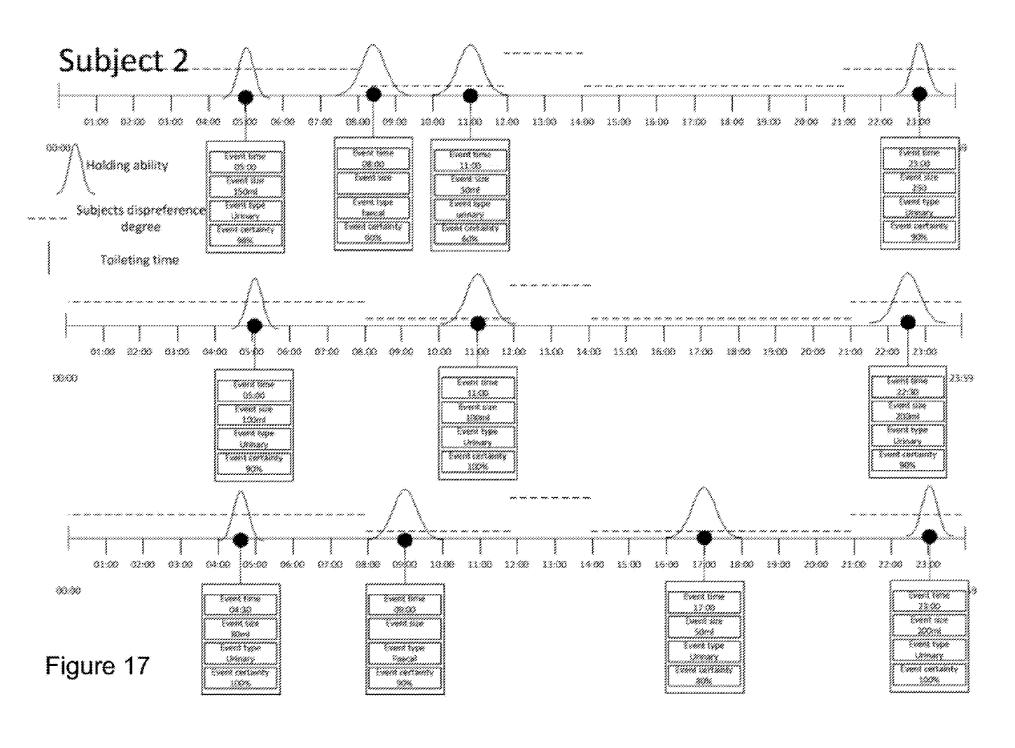

In a variation of the Scenario 3 Procedure, if the permissible number of aid changes is pre-defined (i.e. n is defined in Scenario 3 Procedure) then only step 3 of the Scenario 3 Procedure is required. Step 4 (2006) may utilize an iterative approach (e.g. hill climbing, genetic algorithms, etc.) or an exhaustive search to find the time indicators that maintain a risk of leakage less than 60%. Those time indicators are utilized in the optimised Toileting Schedule generated by processing means 1002. FIG. 13 shows the risk of leakage ROL (y-axis) with one aid change PC at 22:00 is close to 100%. FIG. 14 shows optimal time indicators for two aid changes PC-1 at 08:00 and PC-2 at 17:15, with the same constraints. However, this toileting schedule may not be considered to satisfy the risk of leakage objective since the ROL percentage reaches approximately 70% after PC-1 and 60% after PC-2. The risk of leakage may be maintained below 60% with 3 aid changes as illustrated in FIG. 15.

### Example

### Scenario 4

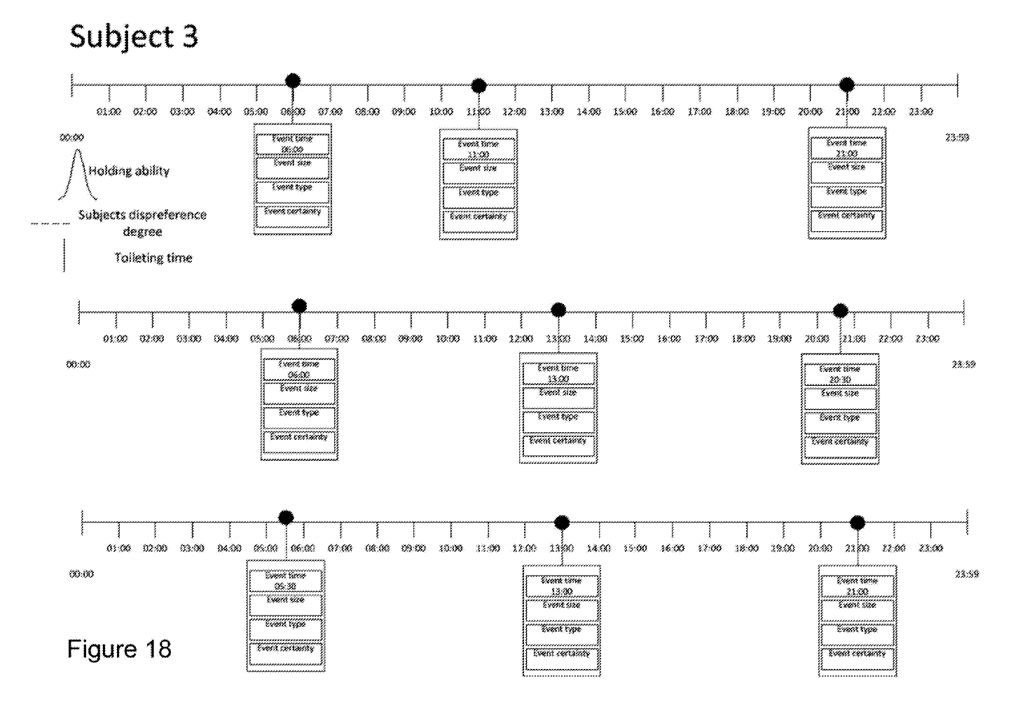

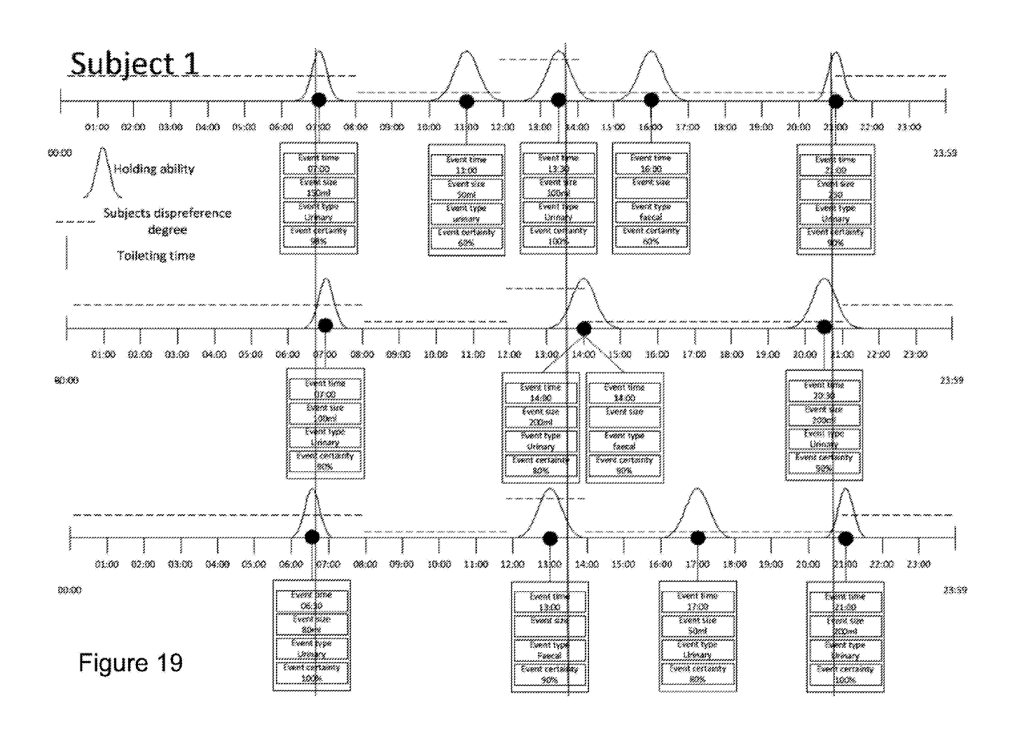

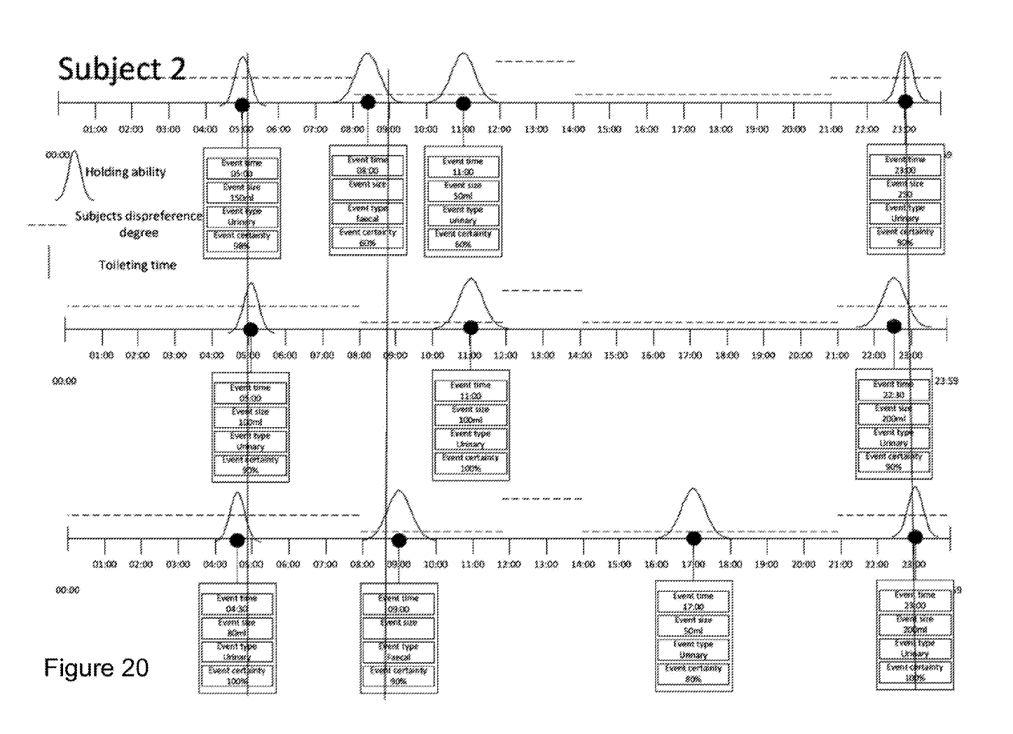

Assume a many-to-many relationship involving 3 subjects with 2 carers. Different numbers of subjects and carers may be treated similarly. FIGS. 16, 17 and 18 illustrate information received as inputs 1001 for each of Subjects 1, 2 and 3 respectively. The inputs include actual event observations, non-preference time periods and holding ability obtained for the subjects over 3 days of observation. Assumptions are that both carers are 100% available at all times and the duration of a toileting procedure is approximately 10 minutes. Table 9 lists the received objectives 1003 and their rakings for the toileting schedule being optimised.

TABLE 9

| Objective function | Rank |
|---|---|
| the Goodness of capturing all the events of the | 1 |
| subjects (generalized form of Equation 17) | |
| Number of events | 1 |
| Non-preferred times for all subjects (generalized | 2 |
| form of Equation 18) | |

A multi-level optimization procedure may be applied because of the relative ranking. In the first level the Goodness of Event and Number of Events objective functions are optimized. In the second level the non-preferred time periods objective function is optimised with the condition that the solution must satisfy the conditions imposed in the first level. In the first level a combination or reduction procedures may be applied, such as:

i(normalized objective value of Goodness of capturing all the events of subjects, normalized objective value of Number of Events)

Many procedures may be adopted for determining an optimised toileting schedule for Scenario 4; these may incorporate a sum function which is used in the function i(value1,value2), and/or multiplication or other functions.

For example in a procedure A, the sum of the normalized values obtained from Equations 21 and 19 may be used as the objective value of the first level. The total objective function is given in Equation 22 below, the solution of which contains the values of a distance function and non-captured event parts. Note that in this example the holding ability, expected event size, expected event type and certainty of the third subject is not known, thus the first objective for this subject is calculated based only on the distance of NPTS and NTS. Equation 22 is calculated for each subject producing a toileting schedule comprising a set of time indicators each of which is associated with a carer, where the objective value (level1ₘᵢₙ) is optimised (in this case, minimised).

Equation 22 may be modified to avoid scheduling toileting procedures with more carers than are necessary allocated to an event e.g. by rejecting or applying a penalty value. Similarly, additional objectives may be incorporated into the procedure to minimise the variability of the Goodness of Events, thereby increasing the likelihood of the derived toileting schedule providing a similar level of quality to all three subjects, in terms of expecting events and scheduling toileting procedures.

$$\begin{matrix}Goodness of capturing all the events of all subjects = 2 {\sum }_{s = 1}^{3} {\sum }_{i = 1}^{n} wf ( Size of {E}_{s , i} ) g ( certainty of {E}_{s , i} ) {\int }_{NTS}^{NPTS} {D}_{s , i} ( t ) d t & Equation 21 \\ Total objective function = N ( Goodness of capturing all the events of subjects ) + Number of Events ) & Equation 22\end{matrix}$$

In the second level a set of time indicators is sought by the processing means for each subject, which minimizes the number of events for all subjects' objective function as given in Equation 22, where m is the number of time indicators in the schedule. The value of the objective function in the first level for the solution sought in the second level must be below level1ₘᵢₙ+margin1. margin1 may be a percentage of level1ₘᵢₙ or a constant or some other value. The third subject is not considered for minimizing the objective function in the second level as the preference degrees for the third subject are not given.

$$Non \text{-} preferred times = \frac{{\sum }_{s = 1}^{3} {\sum }_{j = 1}^{m} {DT}_{s , j}}{m} \qquad Equation 23$$

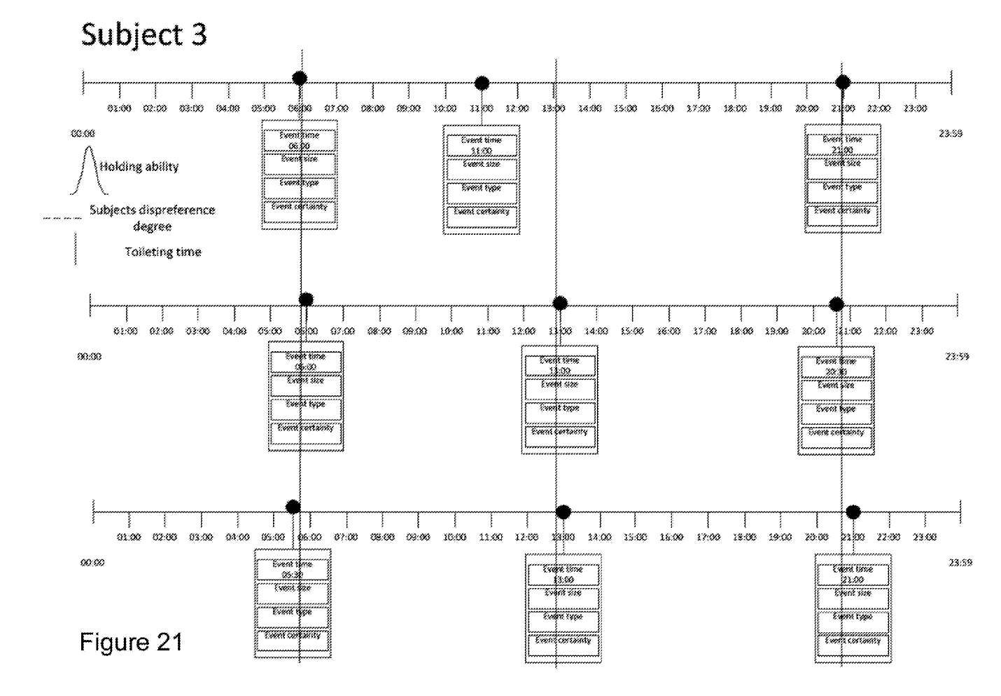

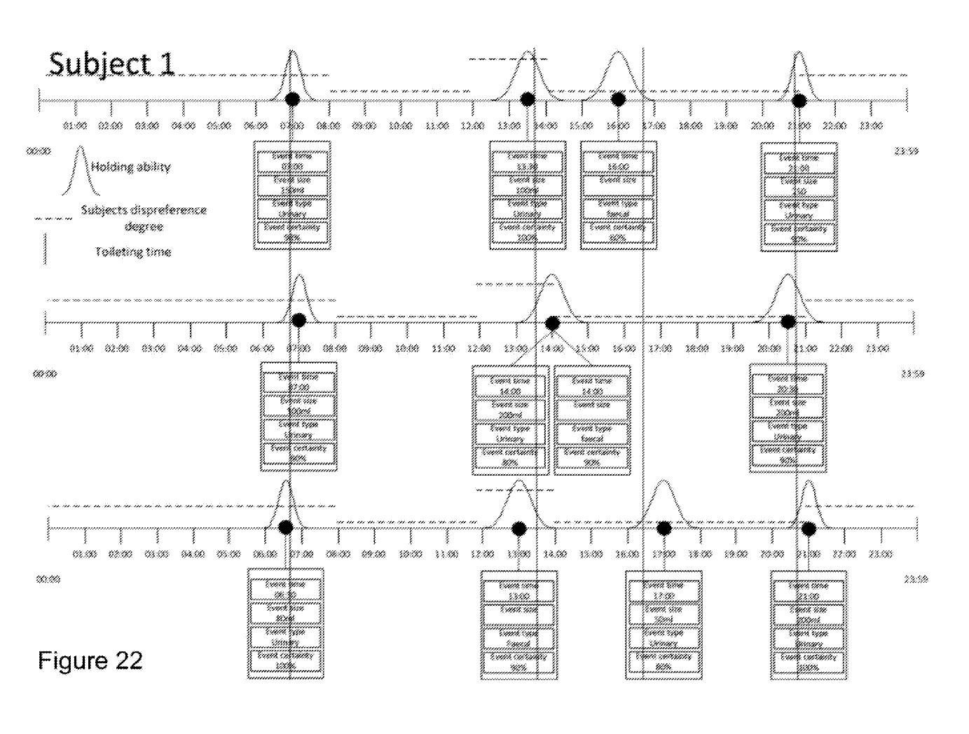

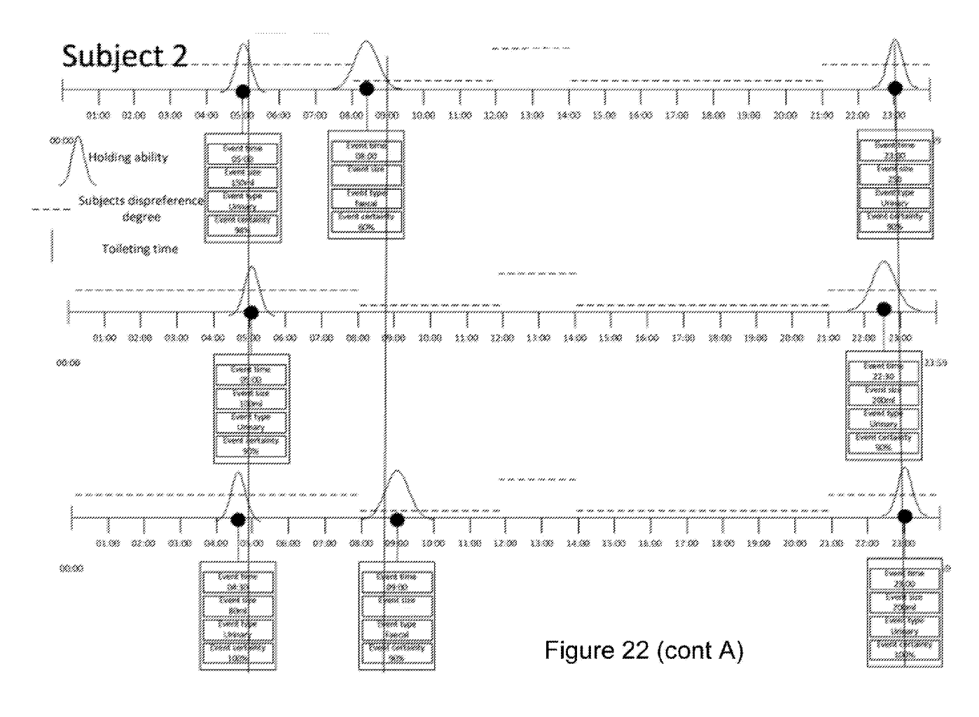

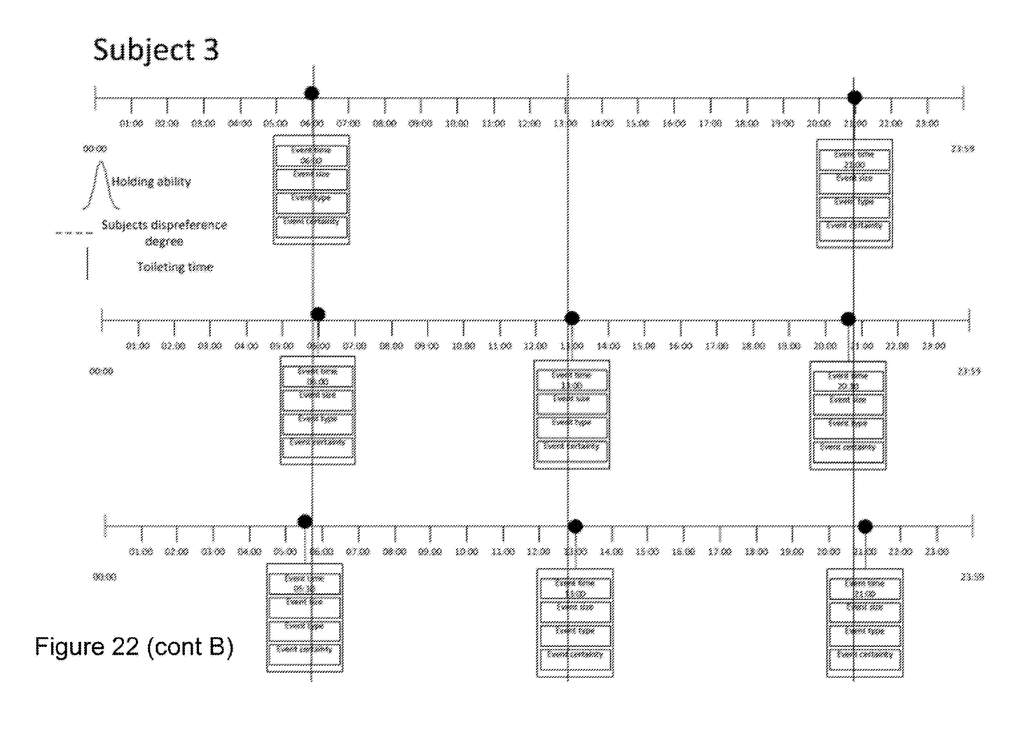

In a procedure B, a set of time indicators for each subject first may be determined using a one-to-one relationship procedure. See Examples for Scenario 1 and Scenario 2. A possible toileting schedule for each subject after applying that procedure is illustrated in FIGS. 19, 20 and 21 respectively. The toileting schedule for each individual subject may then be used to determine an overall schedule, which is dependent on number of available carers. To achieve this, processing means 1002 may exclude the events which are not captured after applying the one-to-one procedure, resulting in the toileting schedule illustrated in FIG. 22.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the claims appended hereto.

1-55. (canceled)

56. An incontinence management system adapted for determining voiding event time indicators representing when a subject's voiding events are expected to occur, the system including:
a) a sensor coupled to a subject that is adapted for detecting an event indicative of the occurrence of a voiding event;
b) a device for:
i. receiving sensor data from the voiding event detection sensor indicative of the occurrence of one or more of the subject's voiding events in a given time period; and
ii. recording time data indicative of the time at which the one or more voiding events occur; and
c) a processor for processing the sensor data and the time data to generate a voiding event time indicator that is optimised in an optimisation procedure with respect to a selection of one or more objectives for determining the voiding event time indicator.

57. The system of claim 56, wherein the sensor is a wetness sensor or a movement sensor or a combination of wetness sensor and movement sensor attached to a subject or a garment worn by a subject.

58. The system of claim 56, wherein the voiding event time indicator comprises one or more of:
a) a single point in time;
b) a set of points in time;
c) an interval specifying a time duration;
d) probability distribution in time; and
e) a function for calculating a time interval between two or more of the voiding event time indicators.

59. The system of claim 56, wherein the voiding event time indicator includes activity time indicators which correspond to times when a voiding event is likely to be experienced by a subject, and wherein the activity time indicator corresponds to a voiding event type selected from the group including but not limited to one or more of:
a. a faecal voiding event;
b. a urinary voiding event;
c. absence of a faecal voiding event;
d. absence of a urinary voiding event; and
e. a combination of a), b), c) and d).

60. The system of claim 56, wherein the voiding event time indicator includes a toileting schedule time indicator used to define a toileting schedule for one or more subjects, the toileting schedule providing a plurality of time indicators for scheduling actions selected from a group including but not limited to:
a. toileting a subject over a commode or receptacle;
b. refreshing or changing an aid worn by a subject;
c. alerting the subject to self-toilet;
d. performing no specific toileting action;
e. alerting a carer that the subject is about to experience a voiding event; and
f. a combination of a), b), c), d) and e).

61. The system of claim 56, further including receiving an existing schedule of time indicators for performing one or more procedures and processing the existing schedule of time indicators with the recorded time data indicative of the time at which the one or more voiding events occur to generate a new schedule of time indicators that is optimised in an optimisation procedure with respect to a selection of one or more objectives for determining voiding event time indicators.

62. The system of claim 56, further including receiving location data indicative of any one or more of carer location, subject location, or bathroom/toilet location and processing the location data and the voiding event time indicator to determine a toileting schedule for any one or more of the carer, the subject and the bathroom/toilet.

63. The system of claim 56, further including receiving a volume of the voiding event and processing the volume of the voiding event and the voiding event time indicator to determine a toileting schedule for any one or more of the carer, the subject and the bathroom/toilet.

64. The system of claim 56, wherein the processor generates toileting schedule data from the voiding event time indicators wherein the toileting schedule data is transmitted to an electronic device including a display device for displaying a toileting schedule.

65. The system of claim 56, further including a feedback device to provide feedback to the subject or a carer before the determined voiding event time or during the occurrence of a voiding event, the feedback provided or controlled by the feedback device comprising any one or more of a recorded human voice, a light, sound or vibration.

66. The system of claim 56, wherein the optimisation procedure includes causing the processor to apply a multi-objective procedure in respect of a plurality of objective functions, wherein the multi-objective procedure includes one or more of the following steps:
a. combining n objective functions representing selected objectives into m new objective functions where m≧n;
b. reducing n objective functions representing selected objectives into m new objective functions where m<n; and
c. treating n objective functions representing selected objectives separately and successively, depending on one or more of a rank order and a relative importance associated with each objective function.

67. The system of claim 66, wherein the multi-objective procedure includes the step of allocating a rank order to at least one of the objective functions to designate a rank order of importance of said at least one objective function relative to others of said objective functions.

68. The system of claim 66, wherein the multi-objective procedure includes the step of allocating a relative importance identifier to at least one of the objective functions representing an importance weighting for said at least one objective function.

69. The system of claim 56, wherein the processor is configurable to use one or more optimisation procedures selected from a group including but not limited to:
a. parallel optimisation in which a multi-objective procedure employs a plurality of objective functions, with or without rank order of importance and with or without relative importance weighting;
b. multi-level optimisation in which there are multiple iterations of the optimisation procedure and each iteration corresponds to a level, the multi-level optimisation beginning with an initial iteration at a first level and ending after a final iteration at a final level, wherein the optimisation procedure optimises at each level a value of one or more objective functions applied at that level, while maintaining a value optimised from a previous level within an acceptable interval defined for said previous level; and
c. hybrid optimisation comprising a combination of parallel optimisation and multi-level optimisation.

70. The system of claim 56, including a stimulation device being adapted to receive a signal indicative of the voiding event time indicator wherein the stimulating device produces a stimulus perceived by the subject before the determined voiding event time to cause stimulation of the subject at a time near the expected voiding event time to raise the subject's awareness.

71. The system of claim 56, including identifying the readiness of a subject for toilet training by determining two or more patterns in the subject's voiding behaviour wherein the pattern is identified by determining if the value of a distance function is less than a threshold.

72. The system of claim 56, wherein the one or more objectives is selected by a user or predefined in the optimisation procedure and includes any one or more of risk of absorbent article leakage, unsuccessful toileting, subject skin problems, pad usage, subject comfort and risk of subject fall.

73. A method for determining voiding event time indicators representing when a subject's voiding events are expected to occur, the method including:
a. receiving sensor data from a sensor coupled to a subject that is indicative of the occurrence one or more of the subject's voiding events in a given time period; and
b. recording time data indicative of the time at which the one or more voiding events occur; and
c. processing the sensor data and the time data to generate a voiding event time indicator that is optimised in an optimisation procedure with respect to a selection of one or more objectives for determining the voiding event time indicator.

74. The method of claim 73, further including generating a toileting schedule for a subject from the voiding event time indicators and transmitting the schedule to an electronic device including a display device for displaying the toileting schedule.

75. The method of claim 73, including receiving the selection of the one or more objectives selected by a user or predefined in the optimisation procedure and includes any one or more of risk of absorbent article leakage, unsuccessful toileting, subject skin problems, pad usage, subject comfort and risk of subject fall.
